(19) United States
(12) Patent Application Publication
(10) Pub. No.: US 2021/0057047 A1
(43) Pub. Date: Feb. 25, 2021

(54) IN-SILICO METHOD FOR DESIGNING A (D)-POLYPEPTIDE LIGAND

(71) Applicant: THE GOVERNING COUNCIL OF THE UNIVERSITY OF TORONTO, Toronto (CA)

(72) Inventors: Michael GARTON, Toronto (CA); Satra NIM, Toronto (CA); Philip M. KIM, Toronto (CA)

(21) Appl. No.: 16/960,792

(22) PCT Filed: Jan. 8, 2019
(86) PCT No.: PCT/CA2019/050026

§ 371 Date: Jul. 8, 2020

Related U.S. Application Data
(60) Provisional application No. 62/614,641, filed on Jan. 8, 2018.

(51) Int. Cl. G16B 35/20; C07K 14/635; C07K 14/64; C07K 14/605; G16B 15/30

(52) U.S. Cl. CPC G16B 35/20; C07K 14/635; A61K 38/00; C07K 14/605; G16B 15/30; C07K 14/64

(57) ABSTRACT

A method for designing in-silico a (D)-polypeptide ligand that binds with a target is provided. The method includes providing a (L)-polypeptide ligand that binds with the target, the (L)-polypeptide ligand comprising one or more (L)-helical region; for each of the one or more (L)-helical region: identifying hotspot residues of the one or more (L)-helical region, that interact with residues of the target; and scanning a (D)-polypeptide database comprising single helix (D)-polypeptide candidates, to determine a single helix (D)-polypeptide match having a residue configuration that matches the hotspot residues of the one or more (L)-helical region; and generating the (D)-polypeptide ligand by combining the single helix (D)-polypeptide match of each of the one or more (L)-helical region.

### TECHNICAL FIELD

The technical field generally relates to a method for designing, in-silico, a (D)-polypeptide ligand.

### BACKGROUND

Proteins and peptides have a number of properties that can make them highly effective as therapeutic agents. These include very precise specificity, high binding affinity, low toxicity, and low risk of drug-drug interactions. Unfortunately, proteins and peptides are susceptible to degradation by proteases and rapid renal clearance. Thus, an array of techniques designed to stabilize peptides and increase their half-life has emerged and is currently driving a rapid expansion in drug candidates. One of the most effective approaches is the incorporation of (D)-amino acids, since biology is peculiarly homo-chiral and constructed almost exclusively from the (L)-enantiomer of amino acids. A useful consequence of this is that (D)-proteins are highly resistant to degradation and have low immunogenicity. There are currently two main existing approaches to engineering proteins with (D)-amino acids. The first approach, retro-inversion (RI), fails if the peptide has a secondary structure, owing largely to the topological properties of helices. The second approach, mirror image phage display (MIPD), is limited to cases where targets have a size of less than −150 residues. This target size limitation together with other difficulties in expression and purification largely precludes membrane proteins, which comprise ˜60% of all therapeutic targets.

Thus, many challenges still exist in the design of (D)-polypeptide ligands.

### SUMMARY

The present description relates to a method for designing in-silico a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target; and
scanning a (D)-polypeptide database comprising single helix (D)-polypeptide candidates, to determine a single helix (D)-polypeptide match having a residue configuration that matches the hotspot residues of the one or more (L)-helical region; and

generating the (D)-polypeptide ligand by combining the single helix (D)-polypeptide match of each of the one or more (L)-helical region.

The present description relates to a method for designing a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target; and
scanning a (D)-polypeptide database comprising single helix (D)-polypeptide candidates, to determine a single helix (D)-polypeptide match having a residue configuration that matches the hotspot residues of the one or more (L)-helical region; and

generating the (D)-polypeptide ligand by combining the single helix (D)-polypeptide match of each of the one or more (L)-helical region.

The present description relates to a computer implemented method for designing in silico a (D)-polypeptide ligand that binds with a target, the method comprising:

providing a (L)-polypeptide ligand that binds with the target, the (L)-polypeptide ligand comprising one or more (L)-helical region to a computing system;

on the computing system, for each of the one more (L)-helical region:
identifying hotspot residues of the one or more (L)-helical region, that interact with residues of the target; and
scanning a (D)-polypeptide database comprising single helix (D)-polypeptide candidates, to determine a single helix (D)-polypeptide match having a residue configuration that matches the hotspot residues of the one or more (L)-helical region; and

generating the (D)-polypeptide ligand by combining the single helix (D)-polypeptide match of each of the one or more (L)-helical region on the computing system.

The present description relates to a computing system for designing in silico a (D)-polypeptide ligand that binds with a target, the system comprising:

a software module configured for:
providing a (L)-polypeptide ligand that binds with the target, the (L)-polypeptide ligand comprising one or more (L)-helical region;
for each of the one more (L)-helical region:
   identifying hotspot residues of the one or more (L)-helical region, that interact with residues of the target; and
   scanning a (D)-polypeptide database comprising single helix (D)-polypeptide candidates, to determine a single helix (D)-polypeptide match having a residue configuration that matches the hotspot residues of the one or more (L)-helical region; and
generating the (D)-polypeptide ligand by combining the single helix (D)-polypeptide match of each of the one or more (L)-helical region.

The present description relates to a non-transitory computer readable medium having instructions stored thereon for designing in silico a (D)-polypeptide ligand that binds with a target, which when executed by a processor causes the processor to perform the steps of:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target; and
scanning a (D)-polypeptide database comprising single helix (D)-polypeptide candidates, to determine a single helix (D)-polypeptide match having a residue configuration that matches the hotspot residues of the one or more (L)-helical region; and

generating the (D)-polypeptide ligand by combining the single helix (D)-polypeptide match of each of the one or more (L)-helical region.

The present description relates to a method for designing in-silico a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target; and
scanning a (D)-polypeptide database comprising single helix (D)-polypeptide candidates, to determine a single helix (D)-polypeptide match having a residue configuration that matches the hotspot residues of the one or more (L)-helical region;
   generating the (D)-polypeptide ligand by combining the single helix (D)-polypeptide match of each of the one or more (L)-helical region; and
   outputting, on a screen for display, a representation of the (D)-polypeptide ligand.

The present description relates to a method for designing in silico a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
generating a (D)-mirror image of the single helix (L)-polypeptide match; and

generating the (D)-polypeptide ligand by combining the (D)-mirror image of the single helix (L)-polypeptide match of each of the one or more (L)-helical region.

The present description relates to a method for designing a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
generating a (D)-mirror image of the single helix (L)-polypeptide match; and

generating the (D)-polypeptide ligand by combining the (D)-mirror image of the single helix (L)-polypeptide match of each of the one or more (L)-helical region.

The present description relates to a computer implemented method for designing in silico a (D)-polypeptide ligand that binds with a target, the method comprising:

providing a (L)-polypeptide ligand that binds with the target, the (L)-polypeptide ligand comprising one or more (L)-helical region to a computing system;

on the computing system, for each of the one or more (L)-helical region:
identifying hotspot residues of the one or more (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
generating a (D)-mirror image of the single helix (L)-polypeptide match; and

generating the (D)-polypeptide ligand by combining the (D)-mirror image of the single helix (L)-polypeptide match of each of the one or more (L)-helical region on the computing system.

The present description relates to a computing system for designing in silico a (D)-polypeptide ligand that binds with a target, the system comprising:

a software module configured for:
providing a (L)-polypeptide ligand that binds with the target, the (L)-polypeptide ligand comprising one or more (L)-helical region;
for each of the one or more (L)-helical region:
   identifying hotspot residues of the one or more (L)-helical region, that interact with residues of the target;
   providing a (D)-mirror image of the one or more (L)-helical region;
   scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
   generating a (D)-mirror image of the single helix (L)-polypeptide match; and
generating the (D)-polypeptide ligand by combining the (D)-mirror image of the single helix (L)-polypeptide match of each of the one or more (L)-helical region.

The present description relates to a non-transitory computer readable medium having instructions stored thereon for designing in silico a (D)-polypeptide ligand that binds with a target, which when executed by a processor causes the processor to perform the steps of:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
generating a (D)-mirror image of the single helix (L)-polypeptide match; and

generating the (D)-polypeptide ligand by combining the (D)-mirror image of the single helix (L)-polypeptide match of each of the one or more (L)-helical region.

The present description relates to a method for designing in silico a (D)-polypeptide ligand that binds with a target, the method comprising:

providing a (L)-polypeptide ligand that binds with the target, the (L)-polypeptide ligand comprising one or more (L)-helical region;

for each of the one or more (L)-helical region:
identifying hotspot residues of the one or more (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the one or more (L)-helical region; and
generating a (D)-mirror image of the single helix (L)-polypeptide match;

generating the (D)-polypeptide ligand by combining the (D)-mirror image of the single helix (L)-polypeptide match of each of the one or more (L)-helical region; and

outputting, on a screen for display, a representation of the (D)-polypeptide ligand.

The present description relates to a method for generating in-silico a (D)-polypeptide database, the method comprising:

generating a mirror image of a (L)-polypeptide database comprising (L)-polypeptides, to obtain a parallel polypeptide database comprising (D)-polypeptides mirror images of the (L)-polypeptides; and

extracting single helix (D)-polypeptides from the parallel polypeptide database, comprising trimming helical regions of the (D)-polypeptides and removing non-helical regions from the parallel polypeptide database, to obtain the (D)-polypeptide database.

The present description relates to a computer implemented method for generating in-silico a (D)-polypeptide database, the method comprising:

providing a (L)-polypeptide database comprising (L)-polypeptides to a computing system;

on the computing system:
generating a mirror image of the (L)-polypeptide database, to obtain a parallel polypeptide database comprising (D)-polypeptides mirror images of the (L)-polypeptides; and
extracting single helix (D)-polypeptides from the parallel polypeptide database, comprising trimming helical regions of the (D)-polypeptides and removing non-helical regions from the parallel polypeptide database, to obtain the (D)-polypeptide database on the computing system.

The present description relates to a computing system for generating in-silico a (D)-polypeptide database, the computing system comprising:

a software module configured for:
generating a mirror image of a (L)-polypeptide database, to obtain a parallel polypeptide database comprising (D)-polypeptides mirror images of the (L)-polypeptides; and
extracting single helix (D)-polypeptides from the parallel polypeptide database, comprising trimming helical regions of the (D)-polypeptides and removing non-helical regions from the parallel polypeptide database, to obtain the (D)-polypeptide database.

The present description relates to a non-transitory computer readable medium having instructions stored thereon for generating in-silico a (D)-polypeptide database, which when executed by a processor causes the processor to perform the steps of:

generating a mirror image of a (L)-polypeptide database, to obtain a parallel polypeptide database comprising (D)-polypeptides mirror images of the (L)-polypeptides; and

extracting single helix (D)-polypeptides from the parallel polypeptide database, comprising trimming helical regions of the (D)-polypeptides and removing non-helical regions from the parallel polypeptide database, to obtain the (D)-polypeptide database.

The present description relates to a (D)-analog of GLP-1, comprising a (D)-amino acid sequence having a sequence identity of 80% or greater to the sequence of SEQ ID NO:1.

The present description relates to the use of the (D)-analog of GLP-1 as defined herein, for the treatment or prevention of diabetes.

The present description relates to the use of the (D)-analog of GLP-1 as defined herein, for the treatment of diabetes.

The present description relates to the use of the (D)-analog of GLP-1 as defined herein, for the treatment or prevention of obesity.

The present description relates to the use of the (D)-analog of GLP-1 as defined herein, for the treatment of obesity.

The present description relates to a (D)-analog of PTH, comprising a (D)-amino acid sequence having a sequence identity of 80% or greater to the sequence of SEQ ID NO:2.

The present description relates to the use of the (D)-analog of PTH as defined herein, for the treatment or prevention of osteoporosis.

The present description relates to the use of the (D)-analog of PTH as defined herein, for the treatment of osteoporosis.

The present description relates to the use of the (D)-analog of PTH as defined herein, for the treatment or prevention of hyperparathyroidism.

The present description relates to the use of the (D)-analog of PTH as defined herein, for the treatment of hyperparathyroidism.

The present description relates to the use of the (D)-analog of PTH as defined herein, for promoting bone growth.

The present description relates to compounds obtained by the method as defined herein.

### BRIEF DESCRIPTION OF THE DRAWINGS

FIG. 1 includes FIG. 1a, FIG. 1b and FIG. 1c. FIG. 1a is a schematic showing the consequence of simple (D) replacement in helical (L)-peptides. FIG. 1b includes charts showing drug target sizes for FDA approved drugs and for targets of drugs subject to preclinical testing or clinical trial. FIG. 1c is a schematic of a method for designing helical (D)-peptides, according to some embodiments of the present description.

FIG. 2 is a schematic illustrating the construction of a (D)-PDB.

FIG. 3 is a schematic illustrating the preparation of GLP-1 (L)-query helices for scanning the (D)-PDB.

FIG. 4 is a schematic illustrating GLP-1 (D)-polypeptide match results and the construction of a (D)-polypeptide.

FIG. 5 is a series of charts and experiments showing the activity and protease degradation of (L)- and (D)-GLP1 polypeptides.

FIG. 6a and FIG. 6b are schematics illustrating PTH (D)-polypeptide match results and the construction of a (D)-polypeptide.

FIG. 6c and FIG. 6d are a series of charts and experiments showing the activity and protease degradation of (L)- and (D)-PTH polypeptides.

FIG. 7 shows default atom levels parameters for each standard amino acid, that may be used for determining matches of hotspot residues in some embodiments of the present description.

FIG. 8 is a table showing amino acid residues grouped by similarity, that can be used in combination with atom levels to increase (D)-match likelihood.

FIGS. 9A to 9C are a schematic representation of a method for designing a (D)-polypeptide, using a mirror image (D)-PDB, according to an embodiment of the present description.

FIGS. 10A and 10B are a schematic representation of a method for designing a (D)-polypeptide, using mirror image (D)-query helical peptides, according to another embodiment of the present description.

FIG. 11 is a schematic representation of the construction of (D)-polypeptides, according to some embodiments of the present description.

FIG. 12 is a schematic illustrating GLP-2 (D)-polypeptide match results and the construction of a (D)-polypeptide.

FIG. 13 is a series of charts and experiments showing the activity and protease degradation of (L)- and (D)-GLP-2 polypeptides.

FIG. 14 schematic illustrating RLN (D)-polypeptide match results and the construction of a (D)-polypeptide.

FIG. 15 is a series of charts and experiments showing the activity and protease degradation of (L)- and (D)-RLN polypeptides.

### DETAILED DESCRIPTION

### Definitions

The expression “polypeptide ligand” refers to polypeptides that are capable of interacting with another compound, such as a target. Interaction of the polypeptide ligand with the target can result in a biochemical reaction or can be a physical interaction or association. More specifically, the interaction of the polypeptide ligand with the target may be the direct binding of the ligand with the target. It is understood that the expression “polypeptide ligand” includes (D)-polypeptide ligands and (L)-polypeptide ligands. (D)-polypeptide ligands consist of, or include, chiral residues having D-, (+)-, or d-chirality. (L)-polypeptide ligands consist of chiral residues having L-, (−)-, or 1-chirality. In other words, (D)-polypeptide ligands as defined herein may be polypeptide ligands that include at least one (D)-amino acid, with the remainder being (L)-amino acids. (D)-polypeptide ligands may preferably be polypeptide ligands that consist of (D)-amino acids. (L)-polypeptide ligands are polypeptide ligands that consist of (L)-amino acids.

It should also be understood that the expression “polypeptide ligand” includes post-expression modification of polypeptides. For example, polypeptides that include the covalent attachment of glycosyl groups, acetyl groups, lipid groups and the like are encompassed by the term polypeptide. Non-limiting examples of polypeptide ligands include glucagon (or glucagon-like peptide-1-GLP-1), calcitonin, parathyroid hormone (PTH), thymorin, teduglutide, pramlintide/amylin, sermorelin, or lucinactant, or a (D)-analog thereof that can be designed by the methods of the present description.

The term “region thereof”, as used herein, refers to a part of a polypeptide sequence, or a polypeptide of any length, that may include for example less than 5%, 10%, 20%, 30%, 40%, 50%, 60%, 70%, 80%, 90%, 95% or more of the polypeptide sequence of a full-length reference polypeptide. In some scenarios, the region can be a region that is functional (e.g. retains the activity of the complete polypeptide or polynucleotide).

The expression “target”, as used herein, refers to a biological molecule of interest, including nucleic acids, proteins (intracellular, transmembrane, extracellular), amino acids, polypeptides, fragments thereof, and the like. Targets may be, for example, receptors, enzymes, binding proteins, antibodies or polypeptides of known or unknown function. The target that interacts with a polypeptide ligand can be present on the surface of a cell or can alternately be an intracellular or extracellular target. The target may for example be a (L)-polypeptide target, such as the GLP-1 receptor (GLP1R) or the PTH receptor (PTH1R), or again the GLP-2 receptor or the Relaxin (RLN) receptor.

The term “helix” or “helical region”, as used herein, refers to a coiled, helical, or spiral, configuration of a protein, polypeptide, peptide, or region thereof, in which successive turns of the helix are held together by hydrogen bonds. Helices may include (L) or (D)-residues and their number of residues may vary. In some scenarios, helices may include between 4 to 50 residues. In some scenarios, a helix may include about 10 residues. It should be understood that helices may also include several unstructured residues adjacent to one or both extremities of the coiled, helical or spiral configuration (for example, helices may include one, two, three or more unstructured residues adjacent to one or both extremities of the coiled, helical or spiral configuration). The term helix includes right handed and left-handed helices. The term also includes, but is not limited to, α-helices, 3₁₀ helices and pi helix (or 7-helix).

The expressions “unstructured”, “nonhelical” and “nonhelical region” refer to a polypeptide or a region thereof without any specific 3D configuration. Unstructured or nonhelical regions may be intrinsically disordered. Such unstructured or nonhelical regions may include linker regions, N-terminus and C-terminus of a ligand polypeptide. It is understood that unstructured or nonhelical regions may include (L) and/or (D)-residues, and that the number of residues may vary.

The terms “residue” or “amino acid residue” or “amino acid” are used interchangeably herein to refer to an amino acid that is incorporated into a protein, polypeptide, peptide or region thereof. The residue may be a naturally occurring amino acid in (L)-polypeptides, (L)-peptides or (L)-proteins, or a (D)-amino acid in (D)-polypeptides, (D)-peptides or (D)-protein.

The expression “hotspot residue” refers to a residue in a polypeptide ligand considered to be relevant for the interaction of the ligand with a target and contributing to the formation of a target/ligand complex. Hotspot residues may contribute to target recognition, binding and/or receptor activation. Hotspot residues may be identified from the literature or by alanine scanning mutagenesis either in vitro or in-silico. For alanine scanning mutagenesis, in some scenarios, no standard free energy change (dG) value is used to define hotspots. In other scenarios, a cut-off between 1.0-2.0 kcal/mol may be used. In the Examples of the present description, a dG value above about +1.0 kcal/mol was used to identify hotspot residues. It should however be understood that the dG value for identifying hotspot residues can vary and is not to be limited by the specific value used in the Examples of the present description. It is also understood that hotspot residues may be any type of residue, such as, but not limited to, negatively charged residues, positively charged residues, uncharged residues, hydrophobic residues, hydrophilic residues or ringed residues.

The expression “retro-inverted version” of a (L)-protein, (L)-polypeptide, (L)-peptide or region thereof, as used herein, refers to a (D)-version of the (L)-protein, (L)-polypeptide, (L)-peptide or region thereof consisting of (D)-amino acids in the reversed sequence, and in which the C-terminus and N-terminus are reversed. Similarly, a “retro-inverted version” of a (D)-protein, (D)-polypeptide, (D)-peptide or region thereof refers to a (L)-version of the (D)-protein, (D)-polypeptide, (D)-peptide or region thereof consisting of (L)-amino acids in the reversed sequence, and in which the C-terminus and N-terminus are reversed.

The expression “junction residue” refers to a residue located at the junction of a helical region and an unstructured or nonhelical region. Junction residues may refer to a residue in a helical region of a polypeptide ligand, that may be the last residue of the coiled, helical or spiral configuration immediately adjacent to an unstructured or nonhelical residue, or in some cases, one of the one or two unstructured residues adjacent to an extremity of the coiled, helical or spiral configuration. Junction residues may also refer to a residue in an unstructured or nonhelical region of a polypeptide ligand that may be immediately adjacent to a helical region, or in some cases, one or two residues from the helical region. Backbone atoms of junction residues may provide orientation to adjacent nonhelical regions in relation to helical regions. Junction residues may be any type of residue, such as, but not limited to, negatively charged residues, positively charged residues, uncharged residues, hydrophobic residues, hydrophilic residues or ringed residues. In some scenarios, junction residues may also be hotspot residues.

The expressions “polypeptide database” or “polypeptide library” refer to a database or a library composed of information regarding the structure of peptides, polypeptides and/or proteins, or regions thereof. Information regarding such structures include, but are not limited to, three-dimensional coordinates and experimental information, such as, unit cell dimensions and angles for x-ray crystallography determined structures. A polypeptide database can be any private or publicly available database, such as, but not limited to, the Protein Data Bank (PDB) or any other database derived from the PDB.

The expressions “binding” or “that binds” refer to the ability of a protein, polypeptide, peptide, or region thereof to interact with a target, either specifically or non-specifically, for example by entering in physical or biochemical contact with the target. Interactions between the ligand and the target include, but are not limited to, any covalent or non-covalent interactions. As used herein, the term “binding” may refer to in vivo, in vitro, or in-silico binding. In-silico binding is generally observed with molecular docking assays wherein the strength of a binding interaction which is a ratio of the association rate over the disassociation rate between the ligand and the target can be calculated. Specific examples include, but are not limited to antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, protein/nucleic acid, ligand/cell surface receptor or virus/ligand.

### Method for Designing a (D)-Polypeptide Ligand

In one aspect of the present description, a method for designing in-silico a (D)-polypeptide ligand that binds with a target is provided.

In some embodiments, the method includes providing a (L)-polypeptide ligand that binds with the target, the (L)-polypeptide ligand including one or more (L)-helical region. It should be understood that the (L)-polypeptide ligand is chosen based on its ability to bind with the target. The ability of the (L)-polypeptide ligand to bind with the target can be determined by an analysis of the literature or by experimentations using various techniques such as, but not limited to, GST pull down, immunoprecipitation, affinity chromatography, equilibrium dialysis, gel filtration, enzyme linked immunosorbent assay (ELISA), FACS analysis, or the monitoring of spectroscopic changes that result from binding.

The (L)-polypeptide ligand includes one or more (L)-helical region. The number of (L)-helical regions that can be present in the (L)-polypeptide ligand, and the length of each (L)-helical region can vary. In some scenarios, the (L)-polypeptide ligand may include two (L)-helical regions, each having a certain number of residues. In other scenarios, the (L)-polypeptide ligand may include three, four, five, six, seven, or more (L)-helical regions, having a certain number of residues. In some scenarios, each (L)-helical region may respectively include between 4 to 50 residues. In some scenarios, (L)-helical regions may include about 10 residues.

In some embodiments, the (L)-polypeptide ligand can include one or more (L)-unstructured region (also referred to herein as (L)-nonhelical region). It should also be understood that the number and length of the (L)-unstructured regions can vary, and should not be viewed as limiting.

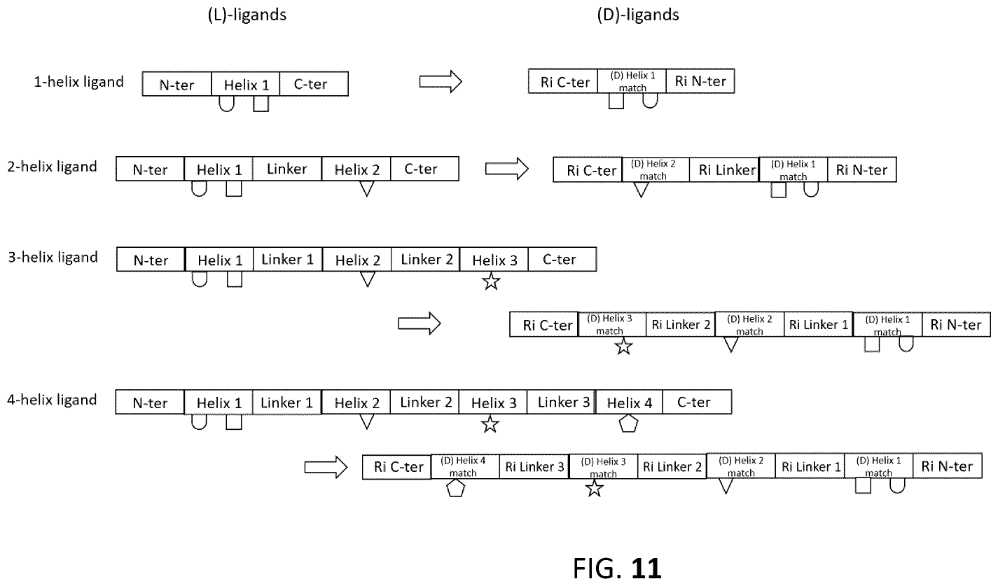

Referring to FIG. 11, non-limiting examples of (L)-polypeptide ligands that include varying numbers of (L)-helical regions (L)-unstructured regions are shown. For example, the (L)-polypeptide ligand can be a 1-helix ligand including one helical region attached to an unstructured N-terminal region and an unstructured C-terminal region. In another example, the (L)-polypeptide ligand can be a 2-helix ligand including two helical regions joined together by an unstructured linker (that can be a flexible linker). The 2-helix ligand can also include an unstructured N-terminal region and an unstructured C-terminal region attached to the first helix and the second helix, respectively. In yet another example, the (L)-polypeptide ligand can be a 3-helix ligand including three helical regions joined together by two (L)-unstructured linkers. The 3-helix ligand can also include an unstructured N-terminal region and an unstructured C-terminal region attached to the first helix and the third helix, respectively. In yet another example, the (L)-polypeptide ligand can be a 4-helix ligand including four helical regions joined together by three (L)-unstructured linkers. The 4-helix ligand can also include an unstructured N-terminal region and an unstructured C-terminal region attached to the first helix and the fourth helix, respectively. It is understood that the (L)-polypeptide ligand can include a larger number of helices and/or unstructured regions. For example, the (L)-polypeptide ligand can include n helical regions (where n is an integer greater than or equal to 1) n−1 unstructured linkers joining the helical regions together, as well as an unstructured N-terminal region and an unstructured C-terminal region. It should also be understood that in some scenarios, the N-terminal region and/or C-terminal region may be helical regions and are not necessarily unstructured. Furthermore, it should be understood that the number of residues of the N-terminal region, C-terminal region or any linker can vary.

In the (L)-polypeptide ligands shown at FIG. 11, the (L)-helical regions are separated from one another by unstructured linkers. However, it should be understood that the (L)-helical regions may be consecutive and need not be separated from one another by any linker. More generally, the (L)-polypeptide ligand can therefore include n helical regions (where n is an integer greater than or equal to 1) as well as p unstructured regions (where p is an integer greater than or equal to 0). In other words, the (L)-polypeptide ligand is not to be limited by the positioning of its (L)-helical regions relative to its unstructured regions.

In some scenarios, the target can be a biological receptor. For example, the target can be the glucagon-like peptide-1 receptor (GLP1R) to which a specific (L)-polypeptide ligand, the glucagon-like peptide-1 (GLP-1), binds. The activation of the GLP1R with GLP-1 is known, for example, to promote insulin secretion and neurogenesis. Therefore, GLP1R activation may be useful for the treatment and/or prevention of diabetes and/or obesity in a subject in need thereof. In another example, the target can be the parathyroid hormone receptor (PTH1R) to which a specific (L)-polypeptide ligand, the parathyroid hormone (PTH), binds. The activation of the PTH1R with PTH, is known, for example, to increase the concentration of calcium in the blood. Therefore, PTH1R activation may be useful for the treatment and/or prevention of osteoporosis and/or hyperparathyroidism, and/or for the promotion of bone growth.

It should be understood that the choice of the target is not limited by the target's size and/or by the presence of transmembrane regions in the target's structure, mainly because the methods described herein are implemented in-silico and do not require synthesizing the target in (D) space, as opposed to MIPD methods.

In some embodiments, the method includes, for each of the one or more (L)-helical region, identifying hotspot residues of the one or more (L)-helical region, that interact with residues of the target. It should be understood that the number of hotspot residues located on each of the one or more (L)-helical region can vary depending on the parameters chosen to define hotspot residues. For example, the (L)-polypeptide ligands shown at FIG. 11 include one or two hotspots for each of their helical regions. The (L)-polypeptide ligands shown at FIG. 11 therefore include between two and 5 hotspot residues. It should however be understood that the (L)-polypeptide ligand can include one hotspot residue, or several hotspot residues, such as 1, 2, 3, 4, 5, 6, 7, 8, or more hotspot residues. Similarly, it should be understood that hotspot residues need not be present in every (L)-helical region of the (L)-polypeptide ligand. In other words, some (L)-polypeptide ligands can include (L)-helical regions that do not include any hotspot residue.

In some scenarios, a scoring function can be used to rank potential binding matches by binding affinity. It should be understood that the term “scoring function”, as used herein, refers to a mathematical expression which is a function of molecular coordinates, and that aims at approximating binding affinity. Scoring functions can be used to rank binding matches with one another, or to distinguish potential binders from non-binders. The result of a scoring function is a number called “score”, which, depending on the scoring function, is to be either minimized or maximized.

It should be understood that hotspot residues can be identified by analyzing the (L)-polypeptide ligand structure obtained from the literature or by experiments, using techniques such as, but not limited to, NMR spectroscopy, X-ray crystallography and/or homology modeling. Alanine scanning mutagenesis experiments can also be performed to identify hotspot residues. Hotspot residues may also be identified on a (L)-polypeptide ligand structure or conformation corresponding to the (L)-polypeptide ligand bound and/or unbound to the target.

In some embodiments, the method includes, for each of the one or more (L)-helical region, scanning a (D)-polypeptide database to determine a single helix (D)-polypeptide match having a residue configuration that matches the hotspot residues of the one or more (L)-helical region. In some scenarios, the (D)-polypeptide database may include single helix (D)-polypeptide candidates. The (D)-polypeptide database can be generated beforehand and accessed for scanning as the in-silico method of the present description is implemented.

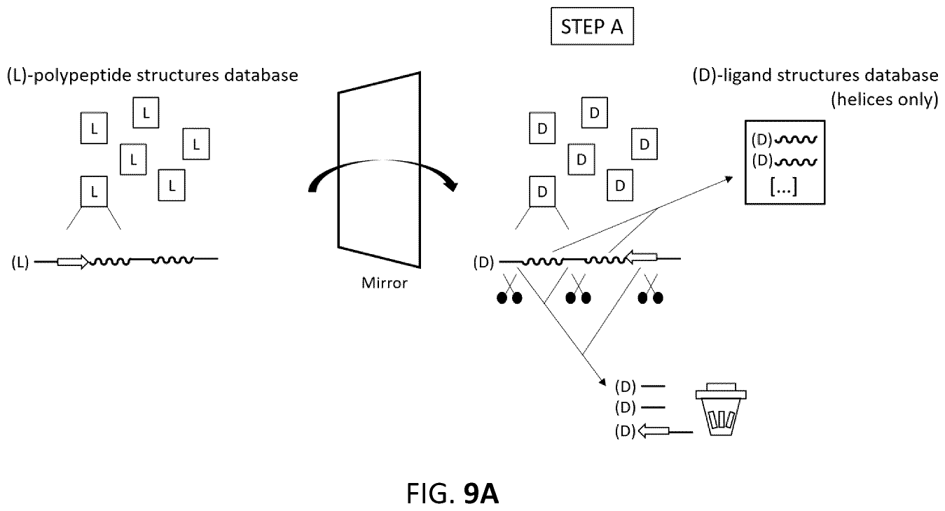

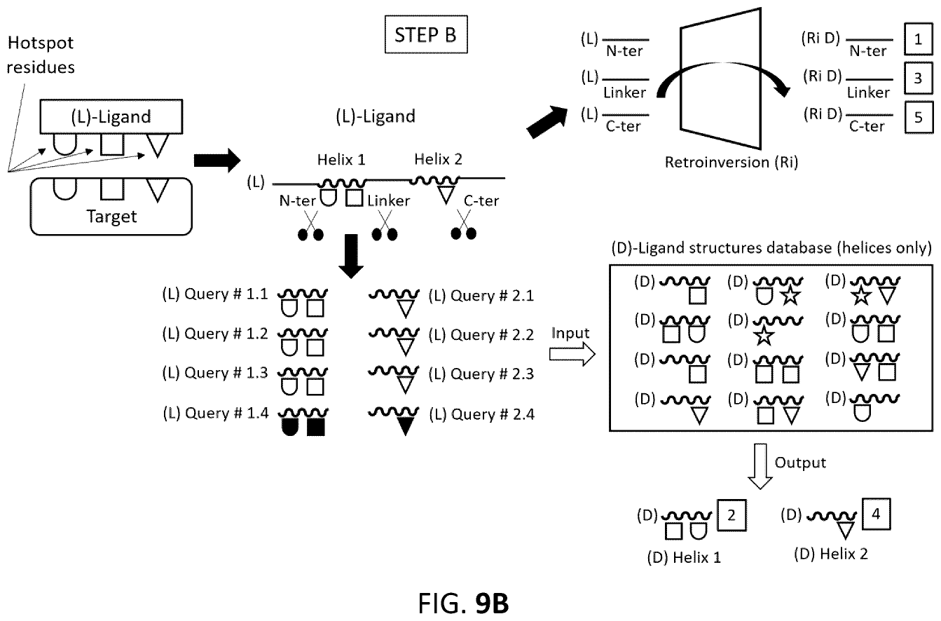

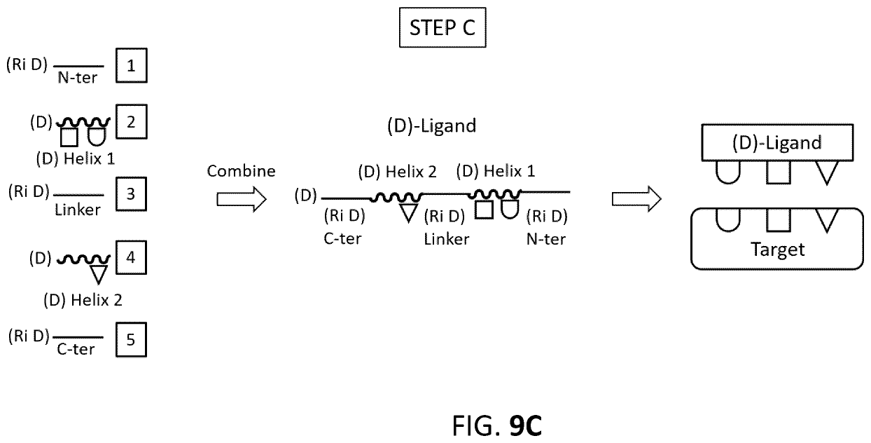

Referring to FIG. 9A, Step A depicts one example of generating a (D)-polypeptide database, based on a (L)-polypeptide database. The (D)-polypeptide database can be generated as part of the method or generated beforehand and accessed as the method is implemented. As seen on FIG. 9A, a mirror image of the (L)-polypeptide database can be generated in-silico. Generating the mirror image of the (L)-polypeptide database can for example include providing a mirror image of each protein, polypeptide and peptide present in the (L)-polypeptide database, to obtain a (D)-polypeptide library (or a parallel polypeptide database).

Still referring to FIG. 9A, the parallel polypeptide library can then be further processed in-silico to single out the (D)-helical regions. In other words, single helix (D)-polypeptides can be trimmed or extracted in-silico and all non-helical parts and non-peptide molecules can be removed from the parallel polypeptide database. For example, every non-protein molecule such as DNA, solvent and ions can be removed from each file of the (L)-polypeptide database before being flipping along one of its axes (i.e., the x, y or z-axis) to create a mirror image, or (D)-version, of each file. Then, nonhelical regions, such as unstructured regions and beta-sheet/strand structures are removed to isolate single helix (D)-polypeptide candidates and generate the (D)-polypeptide database. The (D)-polypeptide database thereby obtained includes single-helix (D)-polypeptide candidates and can be scanned to determine one or more (D)-polypeptide match among the (D)-polypeptide candidates.

It should be understood that the (L)-polypeptide database can be any private or publicly available database that includes protein structures, such as, but not limited to, the Protein Data Bank (PDB) or any other database derived from the PDB. It should also be understood that any file from the (L)-polypeptide database can be flipped along any axis (x, y, z) to obtain a mirror image, or (D)-version, of the file. It should also be understood that the removal of non-protein molecules, the flipping of each (L)-polypeptide database file and the removal of the nonhelical regions to isolate single helix (D)-polypeptide can be performed in any order. For example, the nonhelical regions can be removed from each of the (L)-polypeptide database file before it is flipped to create a mirror image, or (D)-version, file of each single (L)-helical region, then every non-protein molecule can be removed. In another example, the removal of the nonhelical regions can be performed once a (D)-polypeptide match is determined after scanning over a (L)-polypeptide database wherein each file is flipped to create a mirror image, or (D)-version, and non-protein molecules are removed.

Now referring to FIG. 9B, the determination of the various regions of the (D)-polypeptide ligand is shown at Step B. As a (L)-polypeptide ligand is identified, along with its hotspot residues, the (L)-polypeptide ligand can be broken down into one or more single (L)-helical regions and, if present, one or more (L)-unstructured regions. In the example shown, the (L)-polypeptide is broken down into two (L)-helical regions (Helix 1 and Helix 2), a linker region, a C-terminal region and an N-terminal region. For each (L)-helical region, (L)-query helices can be generated by mutating the hotspot residues, which can be mutated back to the original residue in a (D)-polypeptide match without compromising the (D)-polypeptide match structural integrity. In some scenarios, the mutation of a hotspot residue may retain and/or improve the interaction of the (L)-polypeptide ligand with the target and may not be mutated back to the original residue in a (D)-polypeptide match. The (D)-polypeptide database can then be scanned with the (L)-query helices for each of the one or more (L)-helical region. During a scan of each for the one or more (L)-helical region with their respective (L)-query helices, the determination of a single helix (D)-polypeptide match can be based on a match between the residue configuration of a single helix (D)-polypeptide candidate and the configuration of the hotspot residues of the one or more (L)-helical region. For each (L)-helical region, one or more (D)-helical analog can therefore be determined by scanning the (D)-PDB using the (L)-query helices. The nonhelical regions can be retro-inverted to obtain a retro-inverted N terminus, retro-inverted C-terminus and retro-inverted linker.

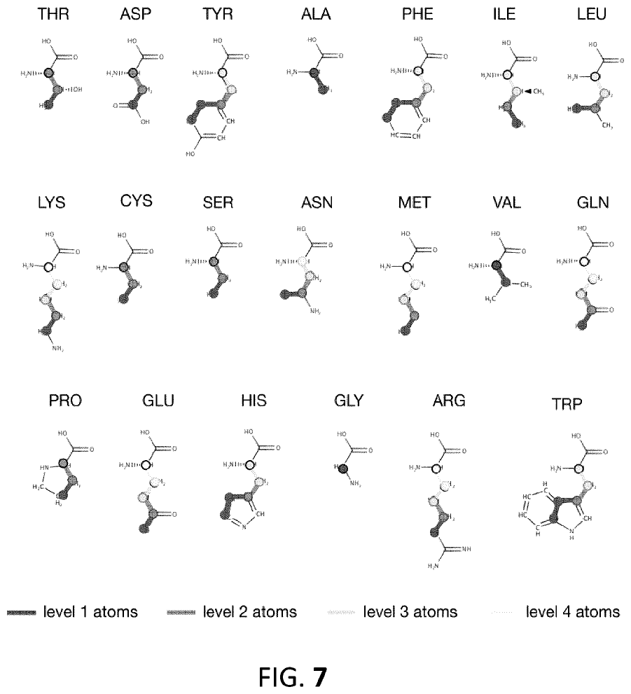

In some scenarios, generating the (L)-query helices can be performed by designating-sets of two or three atoms within each hotspot residues of each of the one or more (L)-helical regions of the (L)-polypeptide ligand, and ranking according to their importance to target interaction, as shown for example in FIG. 7. The highest-level atom sets can be the furthest from the backbone of the (L)-polypeptide ligand, thus closest to its target. Each one or more (L)-helical region with a different atom set combination designated within its hotspot residues can therefore constitute a (L)-query helix (e.g., Queries 1.1 to 1.4 and 2.1 to 2.4), and the ensemble of the (L)-query helices can constitute a query library.

In some scenarios, (L)-query helices with the highest-level atom sets can be used first as an input to scan the (D)-polypeptide database, until a (D)-polypeptide match is determined. A (D)-polypeptide match may be determined when the residue configuration matches the configuration of the hotspot residues of a (L)-query helix, based on the designated atom set of the query. It should be understood that queries with atom sets of any level can also be used first to scan the (D)-polypeptide database. It should also be understood that the method is not limited to the determination of only one (D)-polypeptide match among the (D)-polypeptide candidates. More than one (D)-polypeptide match can be determined, and each can be further processed, as described herein, to improve binding with the target.

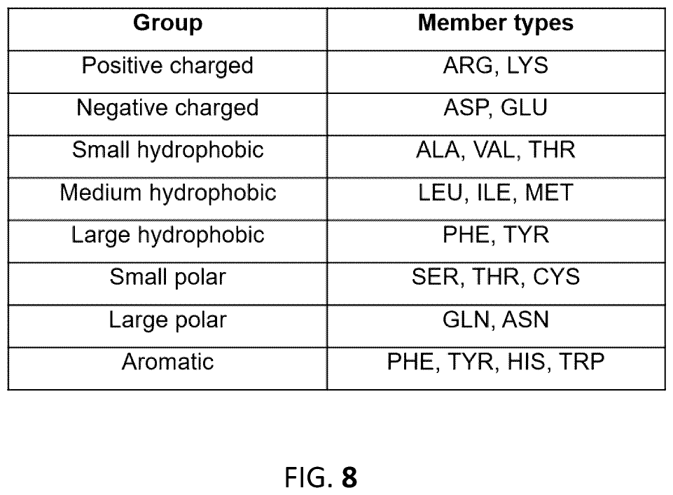

In one embodiment, a query library of (L)-query helices can be generated by mutating one or more hotspot residue, wherein the single helix (D)-polypeptide match is determined comparing the residue configuration with the hotspot residues of the one or more (L)-query helices. In some scenarios, a mutated hotspot residue can be mutated back to the original residue in a (D)-polypeptide match peptide without compromising the (D)-polypeptide match structural integrity. In other scenarios, the mutation of a hotspot residue may retain and/or improve the interaction of the (L)-polypeptide ligand with the target and the hotspot residue may not be mutated back to the original residue. For example, (L)-query helices can be generated by mutating specific hotspot residues of the (L)-polypeptide ligand with any other residue (Queries 1.4 and 2.4), preferably with a chemically similar residue as shown in FIG. 8. Sets of two or three atoms can also be designated within each of the mutated hotspot residues of each of the one or more (L)-helical regions of the (L)-polypeptide ligand and ranked according to their importance to target interaction, as shown in FIG. 7. In some scenarios, the (L)-query helices where a residue is mutated can be used to scan the (D)-polypeptide database when (L)-query helices that do not include mutated residues do not lead to the determination of a (D)-polypeptide match. It should be understood that (L)-query helices where a residue is mutated can also be used first to scan the (D)-polypeptide database.

In one embodiment, the (D)-polypeptide match can be determined by structural alignment of the residue configuration with the hotspot residues of the one or more (L)-helical region. For example, the match quality of single helix (D)-polypeptide candidates can be measured by using the root-mean-square deviation (RMSD) of every atom set combination within the hotspot residues with corresponding atom level combination, if they exist, of a single helix (D)-polypeptide candidate. In some scenarios, the RMSD cut-off is <1.5 Å to determine a (D)-polypeptide match. In some scenarios, the accuracy of the structural alignment is based on the RMSD of the distance between the designated set of atoms within each hotspot residues and equivalent atoms of the single helix (D)-polypeptide candidate. It should be understood that the RMSD cut-off can be set at another value than <1.5 Å, such as, but not limited to, less than 1.0 Å, less than 2.0 Å, less than 2.5 Å, less than 3.0 Å, less than 3.5 Å, less than 4.0 Å, less than 4.5 Å or less than 5.0 Å.

In some embodiments, the method further includes, for each of the one or more (L)-helical region, identifying junction residues that may be immediately adjacent to a (L)-helical region or a (L)-nonhelical region, or in some cases, one or two residues from the (L)-helical region. Once junction residues are identified, the backbone of the junction residues can be positioned to allow specific arrangement of the (D)-retro-inverted version of the one or more (L)-nonhelical region during the generation of the (D)-polypeptide ligand. In one embodiment, the positioning of the backbone of the junction residues includes a first rotation between 170° and 190° about the Cα-Cβ bond axis and a second rotation between 98.5° and 118.5° about the Ca such that Cα-R and Cα-H exchange positions. For example, the positioning of the backbone of the junction residues can include a first rotation of 180° about the Cα-Cβ bond axis and a second rotation 108.5° about the Cα, such that Cα-R and Cα-H exchange positions. The positioning of the backbone of the junction residues is performed such that a (D)-polypeptide match can accept correctly orientated (D)-retro-inverted version of the one or more (L)-nonhelical region.

Once junction residues are identified and their backbone is positioned, the method can further include matching the positioned backbone of the junction residues of the one or more (L)-helical region. It should be understood that with an (L)-polypeptide ligand including one or more (L)-helical regions having junction residues, (L)-query helices can be fixed to include the specific configuration of the positioned backbone (N, O, C & Cα) for the junction residues. Thus, the determination of a single helix (D)-polypeptide match described herein can be based on a match between the residue configuration of a single helix (D)-polypeptide candidate, and the configuration of the hotspot residues of the one or more (L)-helical region, plus the configuration of the positioned backbone of the junction residues. It should be understood that the rest of the determination of a (D)-polypeptide match can remain as described above for (L)-polypeptide ligand including one or more (L)-helical region having junction residues. It should also be understood that a (D)-polypeptide match obtained from a scan including (L)-query helices with junction residues has opposite sequence direction, due to the junction residue backbone rotation about the Cα-Cβ bond axis.

In some embodiments, the method includes generating in-silico the (D)-polypeptide ligand by combining the single helix (D)-polypeptide match of each of the one or more (L)-helical region and optionally (D)-retro-inverted versions of the one or more (L)-nonhelical region. For example, as shown in step C of FIG. 9C, (D)-polypeptide matches for helix 1 and helix 2 of the (L)-polypeptide ligands are combined with the (D)-retro-inverted versions of the N-terminus, C-terminus and flexible linker to generate the full (D)-polypeptide ligand.

Now referring back to FIG. 11, examples of generating the (D)-polypeptide ligand using the (D)-polypeptide match of each of the (L)-helical region and the retro-inverted versions of the unstructured regions are shown. For the 1-helix ligand (i.e., a (L)-polypeptide ligand consisting of only one (L)-helical region, a N-terminus and a C-terminus), the (D)-polypeptide ligand can be generated by combining:
the (D)-retro-inverted version of the C-terminus (which is now the N-terminus of the (D)-polypeptide ligand);
the (D)-retro-inverted version of the N-terminus, (which is now the C-terminus of the (D)-polypeptide ligand); and
the (D)-polypeptide match of the (L)-helical region, having opposite sequence direction to fit with the (D)-retro-inverted versions of the termini.
   For the 2-helix ligand (i.e., a (L)-polypeptide ligand consisting of a N-terminus, a first (L)-helical region, a linker, a second (L)-helical region and a C-terminus, in this order), the (D)-polypeptide ligand can be generated by combining:
the (D)-retro-inverted version of the C-terminus, (which is now the N-terminus of the (D)-polypeptide ligand);
the (D)-polypeptide match of the second (L)-helical region, having opposite sequence direction to fit with the (D)-retro-inverted versions of the C-terminus and linker;
the (D)-retro-inverted version of the linker;
the (D)-polypeptide match of the first (L)-helical region, having opposite sequence direction to fit with the (D)-retro-inverted versions of the linker and the (D)-retro-inverted versions of the N-terminus; and
the (D)-retro-inverted version of the N-terminus, (which is now the C-terminus of the (D)-polypeptide ligand).
   For the 3-helix ligand (i.e., a (L)-polypeptide ligand consisting of a N-terminus, a first (L)-helical region, a first linker, a second (L)-helical region, a second linker, a third (L)-helical region and a C-terminus, in this order), the (D)-polypeptide ligand can be generated by combining, in this order:
the (D)-retro-inverted version of the C-terminus, (which is now the N-terminus of the (D)-polypeptide ligand);
the (D)-polypeptide match of the third (L)-helical region, having opposite sequence direction to fit with the (D)-retro-inverted versions of the C-terminus and second linker;
the (D)-retro-inverted version of the second linker;
the (D)-polypeptide match of the second (L)-helical region, having opposite sequence direction to fit with the (D)-retro-inverted versions of the second and first linkers;
the (D)-retro-inverted version of the first linker;
the (D)-polypeptide match of the first (L)-helical region, having opposite sequence direction to fit with the (D)-retro-inverted versions of the first linker and N-terminus; and
the (D)-retro-inverted version of the N-terminus, (which is now the C-terminus of the (D)-polypeptide ligand).
   For a n-helix ligand (i.e., a (L)-polypeptide ligand consisting of a N-terminus, “n” (L)-helical regions, each separated by a linker (“n−1” linkers) and a C-terminus), the (D)-polypeptide ligand can be generated by combining, in this order:
the (D)-retro-inverted version of the C-terminus, (which is now the N-terminus of the (D)-polypeptide ligand);
the (D)-polypeptide match of the (L)-helical region closest to the C-terminus, now having opposite sequence direction to fit with the (D)-retro-inverted versions of the C-terminus and linker closest to the C-terminus;
the (D)-retro-inverted version of the linker closest to the C-terminus;
the succession of (D)-polypeptide matches of the (L)-helical regions and (D)-retro-inverted versions of the linkers, starting from the C-terminus, wherein the (D)-polypeptide matches have opposite sequence direction to fit with the (D)-retro-inverted versions of the linkers;
the (D)-retro-inverted version of the linker closest to the N-terminus;
the (D)-polypeptide match of the (L)-helical region closest to the N-terminus, having opposite sequence direction to fit with the (D)-retro-inverted versions of the linker closest to the N-terminus and the N-terminus; and
the (D)-retro-inverted version of the N-terminus, (which is now the C-terminus of the (D)-polypeptide).

In some embodiments, the method can further include mutating the (D)-polypeptide ligand. It should be understood that mutations may be introduced for various reasons. In some scenarios, mutations may be introduced to remove clashes between the residues of the target and the residues of the (D)-polypeptide ligand generated herein. In other scenarios, mutations may be introduced to generate a greater similarity between the (D)-polypeptide ligand generated herein and the (L)-polypeptide ligand and/or a greater binding affinity between the (D)-polypeptide ligand and the target. In other scenarios, a previously mutated hotspot residue can be mutated back to the original residue in a (D)-polypeptide match peptide without compromising the (D)-polypeptide match structural integrity. It should also be understood that any kind of mutation is allowed, such as, but not limited to, alanine mutation or mutation with a chemically similar residue as shown in FIG. 8. For example, specific residues R12 and Q13 of the GLP-1 (D)-polypeptide ligand generated were found to clash with the target and were therefore mutated to alanine. W3 and H23 were also provisionally mutated to original query residue types, which were respectively lysine and threonine. In another example, residues A6, E18, W19, R20 and N21 of the PTH (D)-polypeptide ligand generated were also mutated.

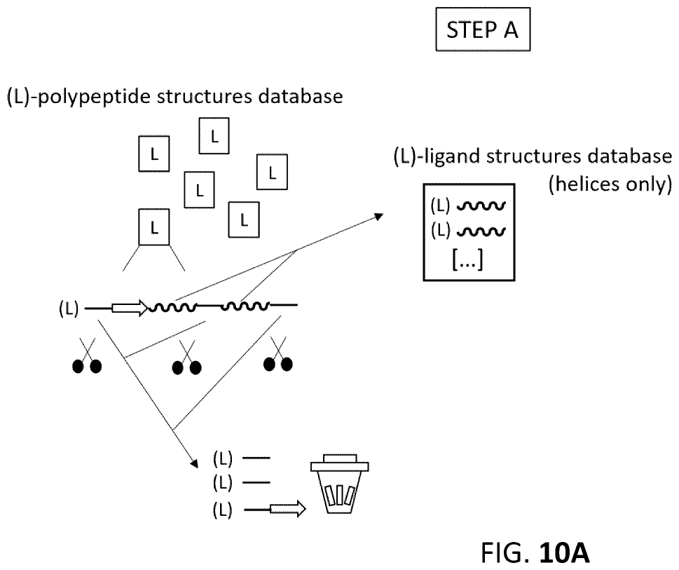

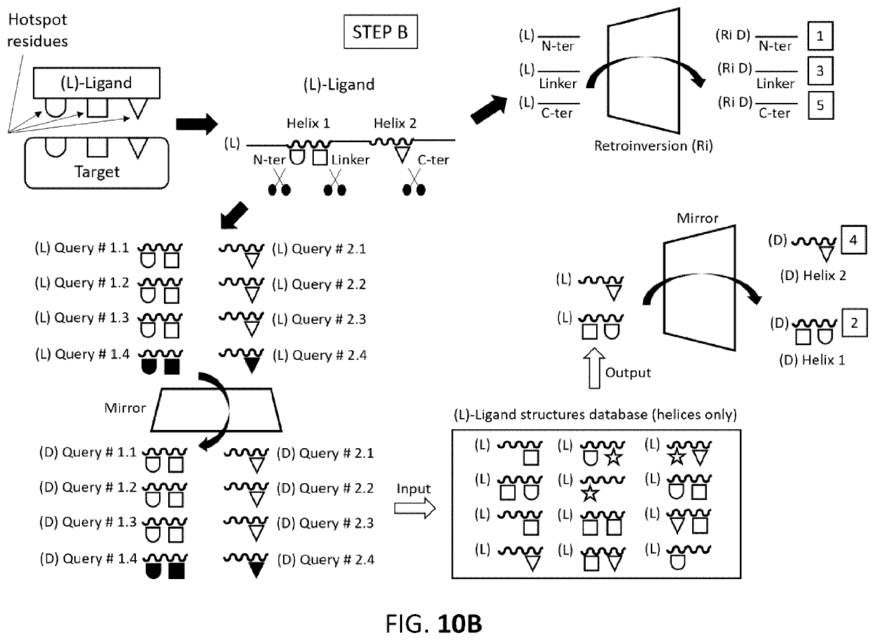

Now referring to FIG. 10A and FIG. 10B., there is provided another embodiment of the method for designing in-silico a (D)-polypeptide ligand that binds with a target. The method makes direct use of the (L)-polypeptide database without generating a mirror image (D)-polypeptide database. After providing the (L)-polypeptide ligand that binds with the target and identifying hotspot residues, the method includes providing a (D)-mirror image of each of the one or more (L)-helical region. It should be understood that the one or more (L)-helical region can be flipped along any axis (x, y, or z) to obtain the (D)-mirror image. The (D)-mirror image of each of the one or more (L)-helical region can be used for constituting a query library of (D)-query helices, that can be used to scan the (L)-polypeptide database. Alternatively, providing a (D)-mirror image of each of the one or more (L)-helical region can be performed after constituting a query library of (L)-query helices, such that the (L)-query helices are converted into (D)-query helices.

As a (D)-mirror image of each of the one or more (L)-helical region is provided, the (L)-polypeptide database can be scanned, to determine a single helix (L)-polypeptide match having a residue configuration that matches the hotspot residues of the (D)-mirror image of the one or more (L)-helical region. In some scenarios, the (L)-polypeptide database may include single helix (L)-polypeptide candidates.

The scanning of the (L)-polypeptide database using the (D)-query helices can be performed in the same way as described above for the (L)-query helices and the (D)-polypeptide database, such that a single helix (L)-polypeptide match is determined for each (D)-mirror image of the one or more (L)-helical region. A (D)-mirror image of each of the single helix (L)-polypeptide match can then be generated.

The (D)-mirror image of each of the single helix (L)-polypeptide can then be combined, optionally with (D)-retro-inverted versions of the one or more unstructured region, to obtain the (D)-polypeptide ligand, as explained above and shown in FIG. 9C.

### EXPERIMENTATION AND EXAMPLES

It should be understood that the examples, values, queries, atom sets, atom levels, rotation angles, ordering of steps, (D)-match determination sequence and any other experimental parameters provided in the “Experimentation and Examples” section below are provided for illustrative purposes only and should not be construed as limiting to the methods for designing (D)-polypeptides of the present description or of the appended claims.

### INTRODUCTION

Biologics are a rapidly growing class of therapeutics that can have several advantages over traditional small molecule drugs. A major obstacle to their development is that proteins and peptides are typically easily destroyed by proteases and thus typically have prohibitively short half-lives in human gut, plasma and cells. One way to prevent or slow down degradation is to engineer analogs from (D)-amino acids, with up to 105-fold improvements in potency reported. A method of peptide-engineering that can overcome limitations of previous methods is described herein. By creating a mirror image of every structure in the PDB, a database of ˜2.8 million (D)-peptides was generated. To obtain a (D)-analog of a given peptide, the (D)-PDB was searched for similar configurations of its critical “hotspot” residues. The method was applied to two peptides that are FDA approved as therapeutics for diabetes and osteoporosis, respectively. (D)-analogs that activate the GLP1 and PTH1 receptors were obtained. The analogs showed similar efficacy and increased half-life, when compared to their natural counterparts.

Using (D)-amino acids as the building blocks for bioactive peptides can dramatically increase their potency. However, simply swapping regular L-amino acids for (D)-amino acids generally alters the peptide surface topology and function is lost. Current methods to overcome this are not generally applicable and exclude the majority of therapeutic targets. By creating a mirror image of all 111,867 protein structures in the PDB, this repository was converted into a (D)-peptide database with 2.8 m D-peptide structures. This D-PDB can be searched to find therapeutically active topologies, demonstrated here by the discovery of novel (D)-peptide GLP1R and PTH1R agonists. of the D-PDB may hold candidates for several therapeutic targets, and potentially contains hundreds of new potent drug leads.

Proteins and peptides have a number of properties that can make them highly effective as therapeutic agents. These properties may include precise specificity, high binding affinity, low toxicity, and low risk of drug-drug interactions. Their diversity also provides very broad coverage of disease targets. Despite this, there are relatively few peptide drugs approved—around 60—compared to around 1,500 small molecule drugs. One major reason for this is thesusceptibility of proteins and peptides to degradation by proteases and rapid renal clearance (1). Consequently, proteins and peptides often have prohibitively short gut, blood plasma, and intra-cellular half-lives. peptides tend to have low intravenous bioavailability and especially poor oral bioavailability, requiring frequent injections and severely limiting their use. Many peptide drug candidates struggle to progress beyond preclinical experiments due to bioavailability considerations. An array of techniques designed to stabilize peptides and increase their half-life has emerged and is currently driving a rapid expansion in drug candidates (2). These include pegylation, backbone modifications, cyclization, stapling, and lipidation (3). One of the most effective approaches is the incorporation of (D)-amino acids (4, 5).

All amino acids except glycine exhibit chirality and therefore can exist in one of either dextrorotary (D) or levorotary (L) forms—so-called because of their influence on plane-polarized light. (D)-amino acids are occasionally found in nature (e.g. in some venoms, antibiotics, and peptidoglycan cell walls) however this is extremely rare (6). Biology is peculiarly homo-chiral and constructed almost exclusively from the (L)-enantiomer. A useful consequence of this is that (D)-proteins are highly resistant to degradation and have low immunogenicity (7). The fundamental change in backbone—side-chain connectivity and geometry means they are not recognized as proteins by many (L)-proteins—including proteases. Consequently, (D)-proteins are reported to typically have greatly increased gut, blood plasma, and intra-celluar half-lives (8). Better cell penetration has also been reported in some cases (9, 10). This behavior can impart potency improvements of up to five orders of magnitude for (D)-proteins and (D)-peptides, when compared with their (L)-counterparts (11).

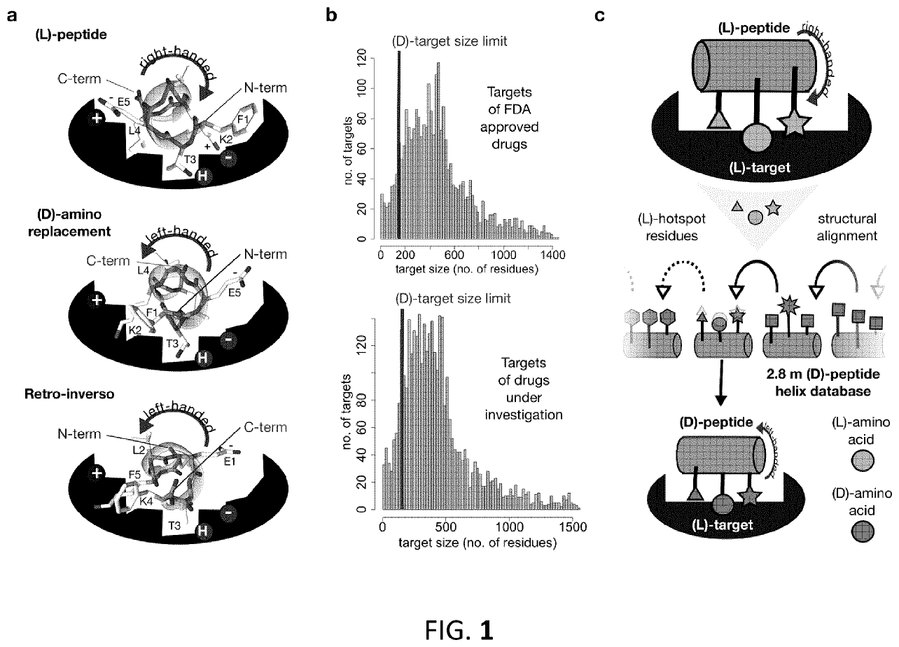

There are two main existing approaches to engineering proteins with (D)-amino acids. Both approaches have significant limitations that preclude application to the majority of known or putative therapeutic peptides and drug targets (12-14). Simply replacing (L) for (D)-amino acids is generally ineffective as side chain orientations with respect to the target are completely altered (15). FIG. 1a shows the consequence of simple (D) replacement in helical (L)-peptides, where a change in side-chain orientation prevents correct binding geometry and typically greatly lowers target binding.

One existing solution to this problem in unstructured peptides is retro-inversion (RI). RI involves reversing the (D)-peptide sequence—flipping the termini, and thus restoring the (L)-amino side chain angles. RI has been used with some success on unstructured peptides (16, 17). The extended (D)-peptides assume side chain topology similar to their parent molecule but with inverted amide peptide bonds. However, retro-inversion usually fails if the peptide has a secondary structure such as a helical structure, owing largely to the topological properties of helices. Indeed, (D)-peptides always adopt left-handed helices (18, 19), while (L)-peptide helices are always right-handed. Left-handed (D)-helices remain left-handed even when the sequence is reversed using RI (FIG. 1a). The resulting topological differences typically greatly lowers binding (12, 15). As approximately 62% of protein-protein recognition in the protein database (PDB) is mediated by helical elements (20) and 80% of FDA approved peptide drugs are helical (14), the majority of therapeutically interesting peptides are therefore inaccessible to the RI technique.

One existing alternative to RI for engineering (D)-amino peptides is mirror image phage display (MIPD). In MIPD, targets are synthesised in (D)-space and used as bait for a randomized (L)-amino peptide library (21). Successful candidate peptides/proteins subsequently made with (D)-amino acids bind the native (L)-protein target with the same affinity as their reverse. Histograms in FIG. 1b show drug target sizes for FDA approved drugs and for targets of drugs subject to preclinical testing or clinical trial. (D)-protein synthesis is currently limited to a target size of ˜150 residues by commercial techniques, although synthesis of up to 312 residues has been reported in an exceptional case (22). This means that MIPD is limited to only a small subset of known targets. Importantly, this target size limitation largely precludes membrane proteins, which include ˜60% of all therapeutic targets (23). Isolated extracellular domains can be made, however, these usually fail to adopt the correct conformation without constraint by the full protein. Transmembrane regions, which are difficult to produce recombinantly, are also often involved in the ligand interaction. Furthermore, agonistic activity requires more than simple binding, making agonist selection difficult with MIPD. In addition to size limitations, many targets require chaperones or obligate hetero-dimeric partners to fold. (L)-chaperones are highly unlikely to specifically recognize a (D)-protein substrate because their topology is very different. Folding is therefore usually precluded (24) although an exception has been demonstrated for DAPA folding by GROEL/ES (22)—thought to proceed using nonspecific hydrophobic interactions.

RI and MIPD limitations mean that the majority of known and putative therapeutic targets are inaccessible to current (D)-peptide engineering techniques. In one aspect of the present description, a method that may overcome at least part of these limitations and may enable the design of helical (D)-peptides to a much broader range of targets is provided. A schematic of the method is shown in FIG. 1c.

[99] The PDB contains over 110,000 naturally occurring and engineered structures. It is therefore a very rich source of information for the rational design of proteins. In some embodiments, the method described herein exploits this resource by creating a mirror image version of the entire repository—thereby rendering every structure of the PDB in (D)-amino acids. The structures can then be further compartmentalized into single helical regions (that is about 2.8 million helices), to form a database called the ‘(D)-PDB’. The (D)-PDB can then be scanned—for example using structural alignment—for residue configurations that match the hotspot residue configurations of therapeutically interesting (L)-peptides (FIG. 1c). Hotspot residues are those identified as contributing significantly to target recognition, binding, and receptor activation. They are a small subset of the full peptide—typically no more than 3 or 4 residues. Finding a structurally equivalent set in the (D)-PDB is therefore highly probable.

Using the glycogen-like-peptide (GLP1) and parathyroid hormone (PTH) as proof-of-concept test cases, D)-helix agonists of the GLP1 and PTH1 receptors using matches discovered in the (D)-PDB were successfully generated. The determination of the (D)-helix agonists of the GLP1 and PTH1 receptors are detailed in the Examples below.

(D)-PDB Construction

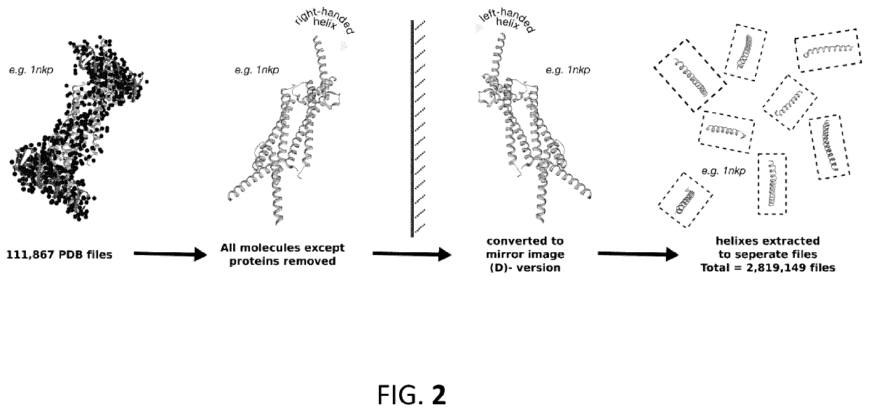

Internal interactions of a protein are identical in its mirror image. This allowed the creation of a parallel protein database composed of (D)-proteins simply by flipping structure files with Cartesian coordinates along the x-axis. Each flipped structure is composed entirely of (D)-amino acids and should fold as the in-silico structure shows when synthesized. A schematic showing (D)-PDB construction is shown in FIG. 2.

After removing any non-protein molecules such as DNA, solvent and ions, each file in the PDB is flipped along the x-axis to create a mirror image version. Non-helical parts of the protein were then removed, and each helix was put into a separate file, totalling more than 2.8 million helix files. This separation ensured that hotspot alignments would only occur on relatively short, contiguous peptide regions. Redundancy was allowed, as even small differences—such as different side chain rotamers—may increase the method power. Since protein regions without secondary structure can effectively be converted to (D) experimentally using RI, such regions were removed from the (D)-PDB. Beta-sheet/strand structures were also removed for simplicity, and because therapeutic peptides tend to be helical, unstructured, or tend to include a combination of helical regions and unstructured regions.

Query Preparation

In a first step, a crystal structure of the functional (L)-peptide was identified or made—or an NMR solution structure of the functional (L)-peptide. A homology model could also be used. It should be noted that homology model effectiveness will likely be highly dependent on the degree of conservation with known structures. Residues critical to target binding and activity can then be identified—often from the literature—by alanine scanning mutagenesis. Ideally this is done experimentally, but with a target bound structure it can also be carried out computationally using techniques such as thermodynamic integration (TI) or free energy perturbation (FEP). It should be understood that the identification of crystal structures, NMR solution structures, or homology models can be either performed in view of implementing the method of the present description or can be retrieved from the existing literature and used as is as a starting point for query preparation.

Once the hotspot residues are identified, various atom sets are designated within each residue. Usually these are pairs of atoms but in the case of ring-containing amino acids such as Phe, Tyr and Trp—a set may include three. Each set is ranked according to its importance to target interaction, with level 1 being the highest. Level 1 usually means the atom pair or triplet furthest from the backbone—and thus closest to the target. It is assumed that if level 1 can be matched, the remaining side chain atoms need not match to be effective. This assumption may increase the chance of finding a match in the (D)-PDB. The other levels are used if level 1 atom pairs do not produce any suitable matches. Lower atom level matches can be used because one of the residue's rotamers—above this level—will usually correctly position the level 1 atoms. Intra-molecular clash can occur between these rotamers and non-hotspot residues in the match. This is identified by full reconstruction of the match, with rotamers that allow correct level 1 positioning. Matches thereby considered non-viable are discarded. Any three atoms of a ring (or rings) can be used to ensure that the correct planarity is represented. Default atom levels used in the Examples herein for each standard amino acid are shown in FIG. 7. It should be understood that other atom sets and levels may be used, as would be known by a person skilled in the art.

Another way that may increase the likelihood of a (D)-PDB match is to group residues by similarity. For example, if a query hotspot is Arg, then matches with both Arg and Lys may be allowed. A (D)-peptide Lys match may be effectively used in the final design or mutated to (D)-Arg with little effect on helix integrity. Similarity residue groupings are shown in FIG. 8 and—with atom renaming—can be used in combination with atom levels to maximise (D)-match likelihood.

In some cases, (L)-peptides of interest have both helical and unstructured regions. Only hotspots in the helical region are used, on the basis that unstructured peptide can be generated by RI—and added in post-processing. To facilitate RI linkage, the last helical residue immediately adjacent to the unstructured region is designated a ‘junction’ residue and included in the alignment (D)-PDB scan. Only backbone atoms (N, O, C & CA) are used for junctions unless the junction is also a hotspot. Using backbone atoms ensures the post-match added RI unstructured peptide will be oriented in the same direction as the (L)-equivalent was. This ensures that correct arrangement of unstructured hotspots in relation to structured hotspots is possible when the unstructured RI region is attached.

For the RI peptide sections to be attached, (D)-matches are chosen to have opposite sequence direction to the L-query. In order to ensure matches have this reversed directionality, junction query backbone atoms are rotated 180° about the CA-CB bond axis. A rotation of 108.5° about the CA—such that CA-R and CA-H exchange positions—is also performed to precisely recapitulate backbone direction. This facilitates extension of the reversed sequence of adjoined unstructured RI regions—where N and C termini are switched. Junction residues thereby allow an RI version of unstructured regions to be attached to (D)-PDB matches. Implementing the 180° rotation step means that D-matches always have the correct sequence direction for RI extension.

### Example 1—GLP-1

GLP-1 is currently of interest as a diabetes mellitus and obesity treatment (25) and was chosen as a first proof-of-concept test case. It involves multiple helices, multiple unstructured regions, negatively charged hotspots, positively charged hotspots, hydrophobic hotspots, ringed hotspots, and a junction-hotspot residue. GLP-1 is a helical GPCR agonist, and this makes engineering a (D)-analog very difficult using conventional methods. There is good availability of structures and hotspot residue information (25), together with a structure for the ligand bound to the extracellular domain of the B-class GPCR (26).

Query Structures for GLP-1

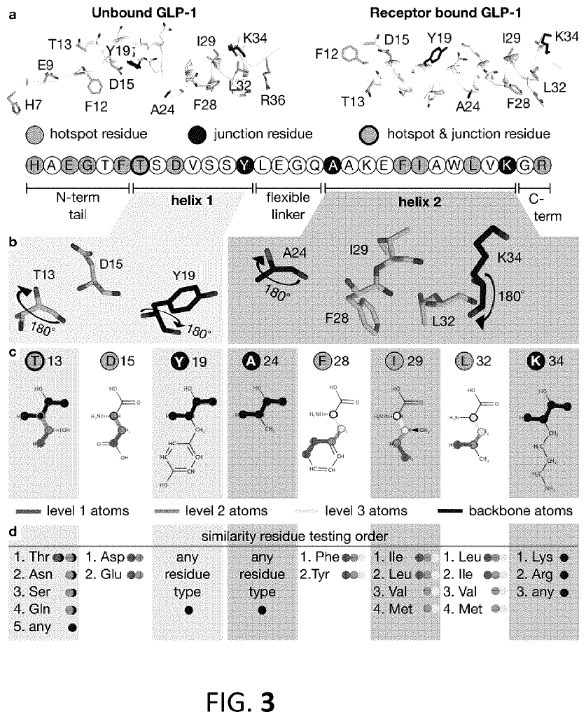

FIG. 3 shows a process of preparing GLP-1 query structures to query the (D)-PDB. Unbound NMR solution structures (PDB ID: 4gzm) and the receptor bound crystal structure (PDB ID: 3iol) were used as starting points. GLP-1 is composed of two helices joined by a four-residue flexible linker. Each helix was set up separately with a view to relinking two matches using the retro inverted linker sequence. Helix 1 runs from T7 to Y13. Helix 2 runs from A18 to K28. In helix 1, T7 and D9 are identified as hotspot residues, while T7 and Y13 act as junction residues. F17, 118 and L20 are the hotspots for helix 2, while A18 and K28 are the junction residues. FIG. 3b shows how hotspot and junction residues are prepared following extraction from their structure. First the junction residue backbone atoms are rotated about the CA-CB axis by 180° and then by 108.5° about the CA along a defined plane. This ensures that a (D)-match can accept correctly orientated RI linker and terminal tail sequence in post-match processing. Following this, six query structures are generated for helix 1, and 27 for helix 2, one for each combination of atom levels (FIG. 3c). In the event of no good match, each of the 33 query structures can be re-run using chemically similar residues. FIG. 3d delineates the order in which each of these was prepared, together with the combinations of atom levels used in each case. K34, while not a definitive hotspot, has been shown to contribute slightly. For this reason, both Lys and Arg were queried before “any”, as a positive charge was slightly preferred.

(D)-Match Output Processing

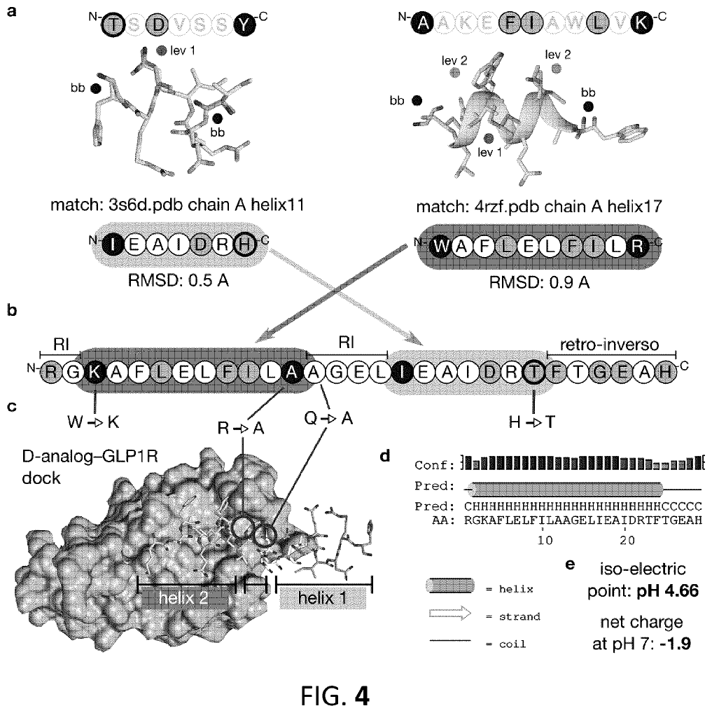

After running each query variant sequentially as outlined in FIG. 3, a number of matches were located in the (D)-PDB. Match quality was measured using the root-mean-square deviation (RMSD) of every atom level combination with corresponding level combinations—if they exist—in every (D)-PDB file. The RMSD cut-off was set for <1.5 Å, although <1.0 Å is ideal if possible. The best match for helix 1 was found in 3s6d.pdb at 0.5 Å, and in 4rzf.pdb at 0.9 Å for helix 2. FIG. 4a shows query-match structural alignments and—together with coloured dots—indicates the successful query variants. Match sequences are reverse ordered due to the junction backbone 180° rotation, allowing RI peptide extension as planned. Both sequences are substantially different to their (L)-query. These matches were then combined with RI unstructured regions to construct the full (D)-analog of GLP-1 (FIG. 4b). A full (D)-analog structure was constructed and docked to the GLP1R ECD structure (FIG. 4c). Residues R12 and Q13 were found to clash with the receptor and were therefore mutated to alanine. W3 and H23 were also provisionally mutated to original query residue types—subject to checks on helix integrity.

The full (D)-analog sequence was checked for helix integrity using PSI-pred (27). FIG. 3d. In addition to the mutations, this was to check that secondary structure is preserved when matched helices are removed from their full protein context. It also provides assurance that the full (D)-analog can fold in the same way as its components. This is necessary in order that the (D)-peptide configuration of hotspot residues closely resembles that of the (L)-peptide and is presented as such to the target. It also highlights any influence that unstructured RI regions may have on the helix or vice versa—such as unwanted structure induced into an RI region by adjoining helix. FIG. 4d shows that (D)-GLP-1 has approximately the same secondary structure profile as (L)-GLP-1. An iso-electric point prediction of pH 4.66—and net charge evaluation of −1.9 at pH 7 for (D)-GLP1 using pepcalc (28) indicates that it has good solubility and thus is suitable for experimental validation.

Experimental Validation of (D)-GLP1

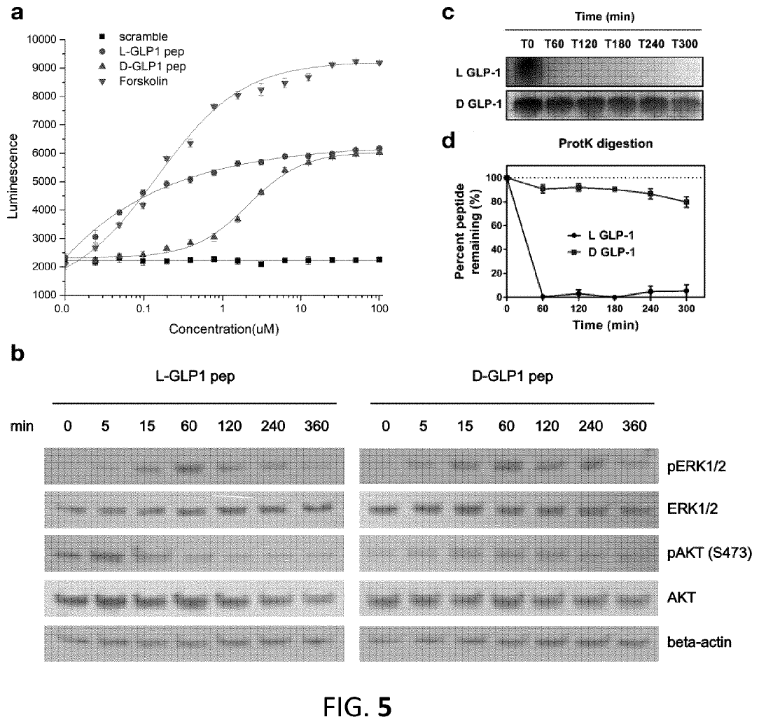

The best candidate was then synthesized from (D)-amino acids and tested for its capacity to activate the GLP1 receptor (GLP1R). Binding of GLP-1 to GLP1R has previously been shown to activate adenylyl cyclase (AC) with consequent production of cAMP, which in turn activates protein kinase A (PKA) to phosphorylate and activate cAMP response element-binding protein (CREB). The ability of (D)-GLP1 peptide to induce activation of GLP1R was investigated and compared the response with native (L)-GLP1 peptide. A stable GLP1 receptor/CRE-luciferase expressing HEK293 cell line was generated and a cAMP-inducible luciferase expression was observed following treatment with Forskolin (FIG. 5a). (L)-GLP1 peptide increased luciferase expression in GLP1 receptor expressing HEK293 cells but was inactive in pCDNA3.1 HEK293 cells. (L)-GLP1 peptide displayed an EC50 value of 59.6 nM with 67.2% efficacy relative to maximum stimulation by Forskolin (FIG. 5a). (D)-GLP1 peptide also increased luciferase expression in GLP1 receptor expressing HEK293 cells (FIG. 5 a). (D)-GLP1 peptide displayed an EC50 value of 2.2 μM with a similar efficacy as the (L)-GLP1 peptide. A scrambled version of (D)-GLP1 was simultaneously tested as a negative control—to account for any non-specific effects—and showed no activity.

To investigate the mechanisms underlying the effects of (D)-GLP1 peptide on GLP1R, the downstream effects of activating GLP1R with (D)-GLP1 peptide were studied. It was investigated whether activation of GLP1R with (D)-GLP1 peptide would induce phosphorylation of ERK1/2 and AKT. In HEK293 cells expressing GLP1R, 10 μM of (L)-GLP1 peptide evoked a robust increase in ERK activation as assessed by the increase in phospho-ERK1/2 (FIG. 5b). The maximum level of phospho-ERK1/2 was achieved around 60 min post-stimulation. (D)-GLP1 peptide at a concentration of 10 μM also activated ERK1/2 evoking a maximum increase of phospho-ERK1/2 around 60 min post-stimulation. The level of phospho-ERK1/2 was sustained after 120 min following (D)-GLP1 treatment while the signal decreased after 60 min with (L)-GLP1.

Resistance to protease degradation is one of the most useful properties of D-peptides generally. Quantitative analysis of the (D)-GLP1 ProtK resistance was carried out and compared to (L)-GLP1. FIGS. 5c and 5d show total loss of (L)-GLP1 in <1 hour, while 80% of (D)-GLP1 can still be detected after 6 hours exposure to ProtK.

### Example 2—Parathyroid Hormone (PTH)

Another test case was selected: parathyroid hormone (PTH) is an FDA approved treatment for osteoporosis delivered by daily subcutaneous injection. Osteoporosis affects approximately 200 million people worldwide but only a fraction receive PTH, partly due to the lack of an oral delivery option. (D)-peptides have shown some oral bioavailability in human trial (29, 30) and thus a (D)-analog of PTH may be of interest. PTH is also of interest for treating hyperparathyroidism (31) and to promote bone growth following fracture (32).

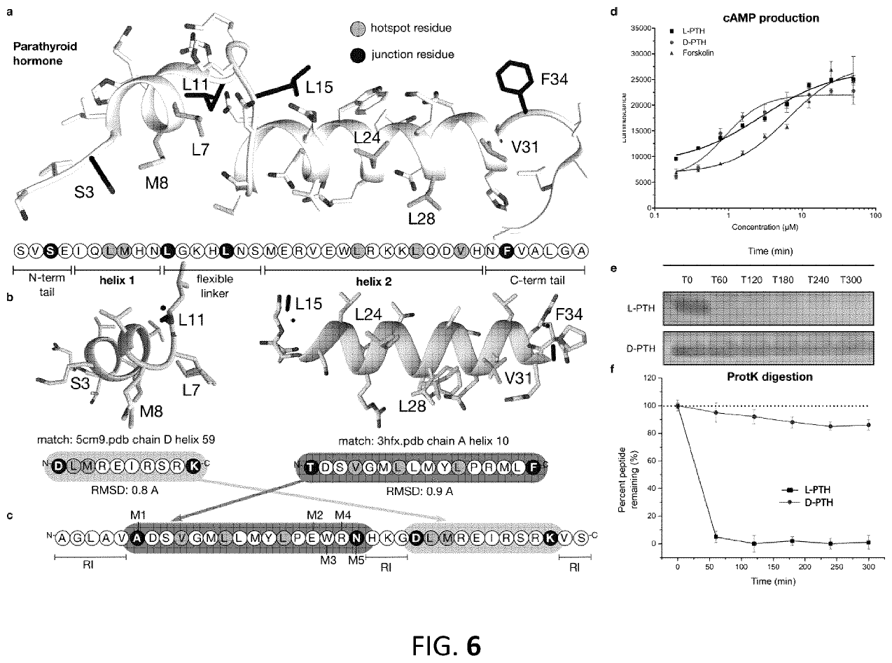

The same process as described for GLP-1 was repeated: crystal structures and hotspot residues were identified from the literature (33, 34). FIG. 6a shows PTH (1-34) with hotspot residues coloured grey and junctions in black, again split into two helices. Helix one hotspots+junctions found a closest (D)-PDB match of 0.95 Å, while helix two was 0.82 Å (FIG. 6b). Reconstruction of the full (D)-peptide using RI for the linker and terminal tails is shown in FIG. 6c. A structural model of the (D)-PTH was constructed and positioned on the receptor to align with hotspot residues. Several mutations were introduced to remove clash and enhance similarity to (L)-PTH (M1-M5).

As with GLP-1 and GLP1R, binding of PTH (residues 1-34) to the parathyroid receptor (PTH1R) has also been shown to activate adenylyl cyclase (AC), triggering cAMP production. This activates protein kinase A (PKA) to phosphorylate and activate cAMP response element-binding protein (CREB). FIG. 6d shows that the (D)-PTH designed here activates PTH1R with a potency and efficacy comparable to (L)-PTH and Forskolin. Protease stability was also calculated and again showed a dramatic difference in degradation rate between the (L)- and (D)-versions (FIG. 6e and FIG. 6f). All of the (L)-PTH is degraded in under 1 hr, while more than 85% of the (D)-analog is still detectable at 6 hours.

### General Applicability

To estimate the general applicability of the method, eight FDA approved peptide drugs that met several criteria were randomly selected. Namely, the criteria included that the peptide drugs were (a) helical; (b) had a target significantly larger than the MIPD (D)-synthesis limit; (c) could benefit from improved half-life; and (d) had an available solved structure. Using the best estimation of hotspot residues, every case had matches in the (D)-PDB with an RMSD of <1.2 Å (Table 1). RMSD measurements of structural similarity for the experimentally validated GLP-1 helix 1 and 2 were 0.5 Å and 0.9 Å respectively. Each match in Table 1 fell into the same approximate range: 0.57-1.15 Å, indicating that this approach is generally applicable. The diversity of conditions also suggests that it could be immediately applied to a wide range of serious conditions including diabetes and cancer.

TABLE 1

| | Trade | PDB | Len. | FDA | | | RMSD |
|---|---|---|---|---|---|---|---|
| Peptide | name(s) | ID | (res) | Apprv. | Condition | t½ | (Å) |
| Glucagon | Glucagon | 1gcn | 28 | 1998 | Hypoglycemia | 15 | 0.92 |
| Calcitonin | Miacalcin | 2glh | 33 | 1975 | Osteoporosis | 58 | 1.04 |
| Parathyroid | Natpara | 1bwx | 39 | 2015 | Hypocalcemia | 180 | 0.88 |
| Hormone | | | | | | | |
| Thymosin | Zadaxin | 2l9i | 29 | 2006 | Hepatitis, Cancer | 120 | 0.57 |
| Teduglutide | Gattex/ | 2l63 | 33 | 2013 | Short bowel | 80 | 0.85 |
| | Revestive | | | | syndrome | | |
| Pramlintide/ | Symlin | 2kj7 | 38 | 2005 | Diabetes type I & | 45 | 0.97 |
| amylin | | | | | II | | |
| Sermorelin | Geref | 5bqm | 31 | 1997 | Weight loss | 12 | 1.08 |
| Lucinactant | Surfaxin | 4esy | 21 | 2012 | Respiratory | n/a | 1.15 |
| | | | | | distress | | |
| | | | | | syndrome | | |

As of 2014, there were over 700 peptides either approved, in clinical trial, or in preclinical development. The current number is likely to be much higher.

Approximately 80% of these peptides are helical, meaning potential candidates for (L) to (D) conversion using methods of the present description already exceed 550. Given the increasing pace of interest in biologics, it is likely that this number will continue to increase rapidly.

### DISCUSSION

Methods of the present description were used to design (D)-peptide analogs of the agonists GLP-1 and PTH that activate GLP1R and PTH1R, respectively. It is a simple and inexpensive method to implement, especially if a starting structure is already available—or can be obtained with reasonable confidence by homology modelling. Otherwise, helical (L)-peptide structures are mostly straightforward to obtain using X-ray crystallography or NMR. Information on hotspot residues can often be sourced from the literature, or otherwise obtained by straightforward alanine scanning mutagenesis experiments.

While (D)-PTH was comparable to (L)-PTH potency and efficacy, (D)-GLP1 potency was ˜40-fold lower than the native peptide, albeit with similar efficacy. Further optimization could involve testing multiple candidates—as only one out of seven (D)-GLP1 candidates produced by the method was tested—and refining with mutagenesis experiments. However, as a proof-of-concept study, the less trivial problem of finding functional (D)-scaffolds was a primary concern. Affinity is a common victim of methods to engineer stability. However, increased stability and longer plasma half-life means that even a large affinity loss can still yield a net improvement in potency. Phospho-ERK experiments indicated that half-life was increased by ˜5-fold. It is well established that GLP1R has rapid internalization and desensitization (35, 36); it is thus likely that this process is responsible for the relatively modest improvement in activity duration, rather than degradation. This was confirmed by proteinase K degradation experiments, which showed improvement in stability for both GLP-1 and PTH. It should be noted that the present description compared the activity of (D)-analogs to the native hormones, and not to the many available analogs that may have higher potency (particularly in the case of GLP1). Therefore, depending on the application, some additional work may be required to optimize (D)-analogs and fully assess their therapeutic potential compared to currently approved solutions. This may involve introducing non-canonical amino acids or chemical modifications.

Non-hotspot residues can vary greatly between the original (L)-peptide and (D)-analogs engineered this way. While not contributing significantly to the interaction, these differences may still adversely affect binding. For instance, bulky or charged (D)-peptide residues may interact with the target in a disruptive manner, especially if that space in the (L)-version is occupied by small or uncharged residues. Mutagenesis could be used to resolve this. GLP-1 was one of the more challenging cases; an agonist consisting of two helices connected by a flexible linker.

(D)-analogs generally avoid some of the limitations of stabilizing methods such as stapling, lipidation, PEGylation (2). These approaches can lead to significant conformational change that can adversely affect their activity. Reduced solubility is another common drawback associated with such approaches. In certain cases, where these limitations are not catastrophic, (D)-analogs could potentially be enhanced using these techniques. Combining approaches is likely to be additive or synergistic in terms of increasing half-life. As such, (D)-PDB matching can be seen as complementing other techniques, rather than competing with them.

Peptide therapeutics are currently undergoing an expansion and the market size is predicted to continue its increase over the next few years (37). The most recent published estimate for the number of peptides in clinical and pre-clinical development is 140 and 500 respectively (3). With approximately 80% of these likely to be helical (14), this means that over 500 of these are potentially immediately applicable for use with the (D)-PDB method. The majority of these are at present prohibited by the limitations of current methodologies. It should be noted that this estimate was published in January 2015 and therefore the current number of peptides in development is likely to now be significantly higher. Several (D)-amino acid containing peptide therapeutics have been approved for use, thus far indicating no inherent toxicity to humans (37).

Materials and Methods

### PDB Preparation

The full latest protein database was downloaded using <rsync-rlpt-v-z--delete--port=33444 rsync.wwpdb.org::ftp_data/structures/divided/pdb/./pdb>. Each file in the database was cleaned to remove any non-peptide components such as water molecules, nucleic acid molecules, metal ions and small molecule drug molecules. For NMR solution structures, only the first model in conformer ensembles was used. Individual helices were then extracted from each of the remaining 111,867 files resulting in 2,819,149 files, one for each helix in the PDB containing a helix plus one non-helix flanking residue at each end. Helices were defined according to information in the PDB file header.

### Hotspot Identification

All necessary hotspot information for GLP-1 and PTH was readily available in the literature from alanine scanning mutagenesis experiments.

### Structural Alignment

Structural alignments were carried out using a program called Click (38). Click was chosen because unlike the majority of structural alignment software, it does not consider sequence order or use sequence alignment. Instead, it uses the molecule Cartesian coordinates to align constellations of points independent of residue order. This is important for identifying the closest matching D-peptide hotspot constellations because their sequence order and/or direction is very often different to the L-peptide query.

### Target Compatibility

Helix matches were assembled using Chimera (39) on the surface of the GLP-1R and PTH1R ECDs (PDB IDs: 3iol & 3c4m). Matched (D)-hotspots were aligned with their corresponding (L)-hotspots. The central linker region was constructed using chimera and the saved coordinate file converted to (D). The linker was also then assembled on the surface of GLP-1R & PTH1R such that it lined up with helix junction residues. Residues that clashed with the target were mutated accordingly.

### Helix Integrity Checking

PSI-PRED (27) was used to predict the likely secondary structure of each candidate. Recalculation was carried out following each mutation to remove target clash and mutations were accepted on the basis that helical structure was predicted. Deviation from helical would have led to mutation to different residues types until helix was maintained and clash removed. Failure to do both means the candidate would be demoted. The web tool PepCalc (28) was used to predict peptide solubility. If poor solubility was predicted, the mutations would be revised, secondary structure checks repeated, and solubility checks rerun. This process would be repeated until all clash, secondary structure, and solubility requirements are satisfied.

### Peptide Synthesis

Both (L)- and (D)-peptides were obtained from Lifetein LLC, (Somerset, N.J.), and were produced by chemical synthesis.

### Cell Lines and Reagents

HEK293 cell line was obtained from the American Type Culture Collection (ATCC; Rockville, Md.). HEK293 cell line was tested for mycoplasma contamination. HEK293 cells were maintained in DMEM (ATCC) supplemented with 10% FBS and 1% pen/strep/glutamine, and the appropriate selection antibiotics when required.

### Library Construction, Amplification and Lentiviral Plasmid Construction

Gaussia Luciferase vector was generated by PCR amplification of the Gaussia Luciferase gene from the pTK GLuc (provided by the Stagljar lab) using primer for insertion of restriction sites (EcoRI and Xmal): Primer forward 5′-GGAACTAACCGGTCGCCACCATGGGAGTCAAAGTTCTGTTTGCC-3′, primer reverse 5′-CAATGCCGAATTCTTAGTCACCACCGGCCCCCTTGATC-3′. The PCR product was digested and cloned into pLJM17 lentiviral vector. The pLJM17 vector contains a CMV promoter and hygromycin for the selection marker.

### Luciferase Assay

HEK293 cells stably expressing hGLP1R and reporter CRE-Gaussia Luciferase construct were trypsinized from subconfluent culture and seeded in a 96-well plate at a density of 5,000 cells per well. Cells were incubated overnight at 37° C. in 5% CO2. Cells were treated with different concentrations of L-GLP1 peptide, D-GLP1 peptide and forskolin. After 6 hours of incubation, 20 uL of cell medium was transferred to a black flat-bottomed 96-well plate. 50 uL of Working solution (Pierce Gaussia-Firefly Luciferase Dual Assay Kit, Thermo Scientific #16181) was added into each well containing cell medium. Immediately after adding the reagent, samples were read using a luminometer with a 480 nm filter.

### Western Blot

HEK293 cells stably expressing hGLP1R were treated with different concentrations of L or D-GLP1 peptides for different time points. Cells were lysed with lysis buffer (50 mM Tris-HCl pH7.4, 1% Nonidet P-40, 150 mM NaCl, 1 mM EDTA, 10 mM Na3VO4, 10 mM sodium pyrophosphate, 25 mM NaF, lx protease inhibitor mixture (Sigma) for 30 min at 4° C. Protein samples were separated on a NuPage Bis.Tris 10% SDS/PAGE gel (Invitrogen) and transferred to PVDF membranes. Transferred samples were immunoblotted with primary antibodies, followed by incubation with horseradish peroxidase-conjugated secondary antibodies (Santa Cruz Biotechnology) and detected using enhanced chemiluminescence (GE Healthcare).

### Protease Stability Assay

Stocks of 20 μM peptide in 200 μL total volume (10 mM Tris-base, 10 mM NaCl, pH 7.4) were supplemented with 5 μM CaCl₂) and 30 uL removed for the untreated T0 sample. Proteinase K (ProtK, Bioshop) was then added to a final concentration of 100 μg/mL. Samples were incubated at 37° C. and 30 μL removed after each time point and protease activity blocked by the addition of 10 mM PMSF (200 mM stock dissolved in isopropanol). Protease inactivated samples were frozen at −20° C. until further use. Digestions were repeated three times. Frozen samples were supplemented with 8 μL sample loading buffer (4× NuPAGE, ThermoFisher Scientific), boiled (50° C.) for 10 minutes, and centrifuged (12 000 rpm, 10 min) prior to loading the gel (12% NuPAGE Bis-Tris (ThermoFisher Scientific) with MES running buffer). Gels were run at 200 V for −35 minutes and stained using Coomassie Brilliant Blue dye. Densitometry of bands was determined using ImageJ software (40) with back ground subtraction. All samples were normalized to their respective untreated sample (T0).

### Circular Dichroism

Secondary structure determination was carried out using a Jasco J-720 spectropolarimeter. Lyophilized peptide powders were dissolved in pure water and CD spectra read immediately. Peptide concentrations were 20 μM for L-GLP1 and 150 μM D-GLP1 in water. Concentrations varied between peptides to enable collection of clear spectra, as peptides generally lacked strong CD signals. Samples were read using a 0.1 cm cuvette pathlength with 3 accumulations per run, 50 nm/min scanning speed. All spectra were background subtracted and converted to mean residue molar ellipticity (MRE) using standard formulas to allow direct comparison between samples of varying concentration and amino acid length. Spectra are reported in the supplemental information (FIG. 9). The D-GLP1 peptide spectra has been inverted to allow for visual comparison to the L-GLP1 peptide spectra.

### Example 3—GLP-2

Another test case was selected: GLP-2. GLP-2 is a gastrointestinal peptide with about 33% sequence homology to glucagon. GLP-2 is a potent intestinotrophic growth factor with therapeutic potential for the prevention or treatment of a number of gastrointestinal diseases, including metabolic endotoxemia, obesity, metabolic syndrome and short bowel syndrome (SBS). GLP-2 can also be used for the treatment of diabetes. GLP-2 involves two helices directly linked with one another, as well as N-terminal and C-terminal regions. There is a need for developing analogs of GLP-2 that can have a longer half-life than GLP-2 and a comparable activity.

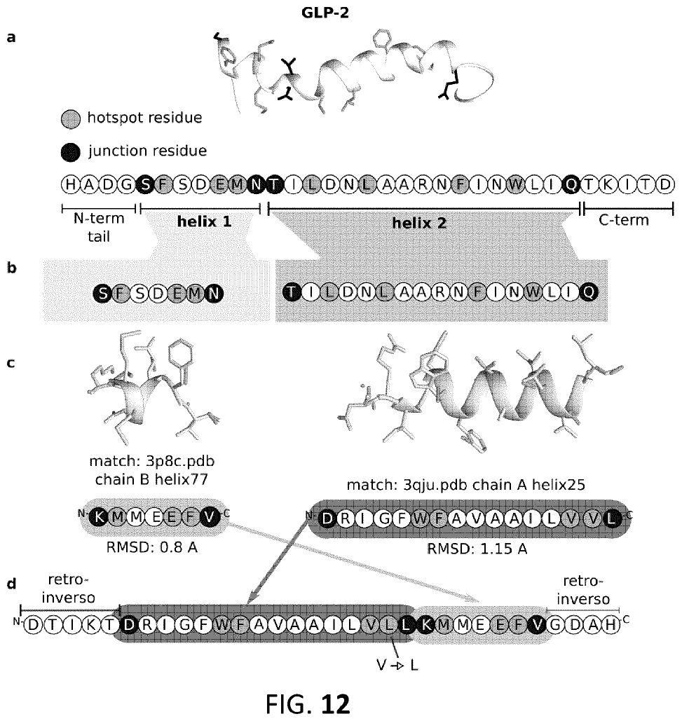

The same process as described for GLP-1 and PTH was repeated: GLP-2 was solved using NMR and hotspot residues were identified from the literature. FIG. 12 shows GLP-2 with hotspot residues coloured grey and junctions in black, again split into two helices. Helix one hotspots+junctions found a closest (D)-PDB match of 0.8 Å, while helix two was 1.15 Å. Reconstruction of the full (D)-peptide analog using RI for the linker and terminal tails is also shown. A structural model of the (D)-GLP-2 was constructed and positioned on the receptor to align with hotspot residues. One mutation was introduced to remove clash and enhance similarity to (L)-GLP-2.

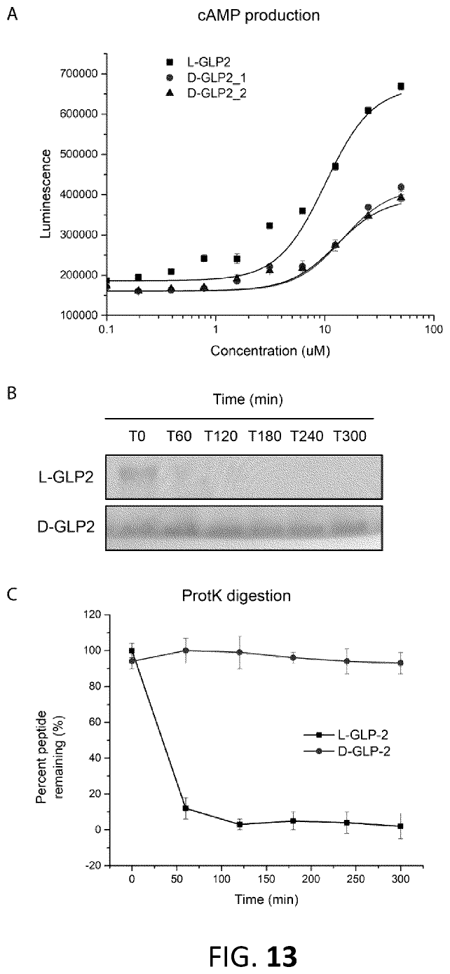

As with GLP-1 and GLP1R, and PTH and PTH1R, binding of GLP-2 to GLP2R has also been shown to activate adenylyl cyclase (AC), triggering cAMP production. This activates protein kinase A (PKA) to phosphorylate and activate cAMP response element-binding protein (CREB). FIG. 13 shows that the (D)-GLP2 designed here activates GLP2R with a potency and efficacy comparable to (L)-GLP-2. Protease stability was also measured and showed a dramatic difference in degradation rate between the (L)- and (D)-versions. All of the (L)-GLP-2 is degraded in about 1 hr, while more than 90% of the (D)-analog is still detectable at 6 hours.

### Example 4—Relaxin (RLN)

Another test case was selected: Relaxin (RLN). RLN is a multifunctional factor that can be used in a broad range of target tissues including several non-reproductive organs, in addition to its historical role as a hormone of pregnancy. For example, Relaxin can be used in the treatment of fibrosis, inflammation, cardioprotection, vasodilation and wound healing (angiogenesis), amongst other pathophysiological conditions. RLN involves one helix, as well as N-terminal and C-terminal regions. There is a need for developing analogs of RLN that can have a longer half-life than RLN and a comparable activity.

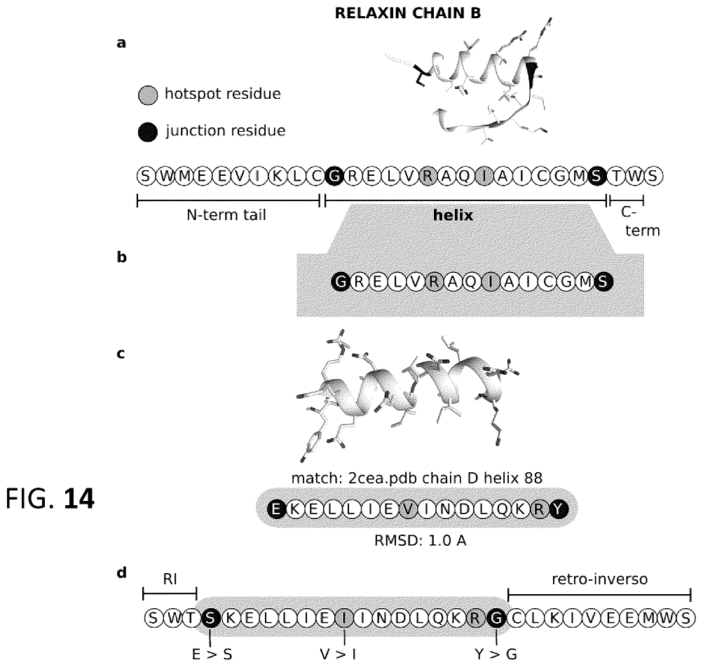

The same process as described for GLP-1 and PTH was repeated: crystal structures and hotspot residues were identified from the literature. FIG. 14 shows RLN with hotspot residues coloured grey and junctions in black. Helix hotspots+junctions found a closest (D)-PDB match of 1.0 Å. Reconstruction of the full (D)-peptide analog using RI for the linker and terminal tails is also shown. A structural model of the (D)-RLN was constructed and positioned on the receptor to align with hotspot residues. One mutation was introduced to remove clash and enhance similarity to (L)-RLN.

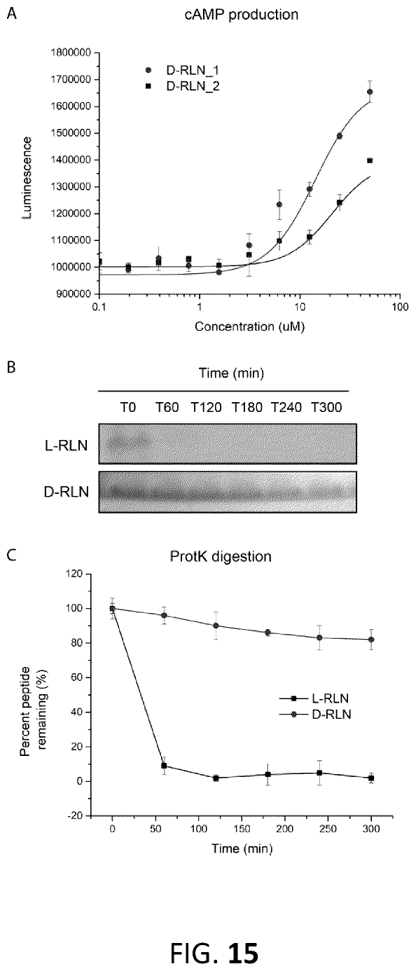

Binding of RLN to its receptor has also been shown to activate adenylyl cyclase (AC), triggering cAMP production. This activates protein kinase A (PKA) to phosphorylate and activate cAMP response element-binding protein (CREB). FIG. 15 shows that the (D)-RLN designed here activates the RLN receptor with a potency and efficacy comparable to (L)-RLN. Protease stability was also measured and showed a dramatic difference in degradation rate between the (L)- and (D)-versions. All of the (L)-RLN was degraded in about 1 hr, while about 90% of the (D)-analog was still detectable at 6 hours.

### Example 5—in Silico Design of Other D-Polypeptide Analogues

Using the in-silico method described herein, in the same manner as described in Examples 1-4, the D-polypeptide structures shown in Table 2b below were designed in silico to be D-match sequences of the L-polypeptide sequences shown in Table 2a below. The L-polypeptide sequences shown in Table 2a below are known to bind to the corresponding targets listed in Tables 2a and 2b.

TABLE 2a
seven (7) L-polypeptides

| | L- | |
|---|---|---|
| Target | Structures | L-Sequence |
| Zika Virus | 5IRE | MAVLGDTAWDFGSVGGA |
| (Zika | | LNSLGKGIHQIFGAAFK |
| treatment) | | |
| Dengue Virus | 3J27 | MAILGDTAWDFGSLGGV |
| (Dengue | | FTSIGKALHQVFGAIY |
| Treatment) | | |
| PACAP | 1GEA | HSDGIFTDSYSRYRKQ |
| (migraine | | MAVKKYLAAVLGKRYK |
| treatment) | | |
| PYY | 2DF0 | YPIKPEAPGEDASPEEL |
| (treatment of | | NRYYASLRHYLNLVTRQ |
| obesity) | | RY |
| FOX04 | 1El7 | GRKKRRQRRRPPPRKGG |
| (promoting | | SRRNAWGNQSYAELISQ |
| senescent cell | | AIESAPEKRLTL |
| viability) | | |
| GRS (cancer | 2PME | MYTVFEHT |
| treatment) | | |
| Glucagon | 1GCN | HSQGTFTSDYSKYLDSR |
| (hypoglycemia | | RAQDFVQWLMNT |
| treatment) | | |

TABLE 2b
seven (7) D-Match sequences

| | D-Match | D-Match |
|---|---|---|
| Target | Structures | Sequences |
| Zika | 3PVY, | REEVYEIFHAQHGTVRSLAFLA |
| Virus | 4XDN, | TVSGFFERVWTGEMVVVAVC |
| | 2HJF | |
| Dengue | 4TQU, | -DVDKTWDEYQ |
| Virus | 3B8B | -SPLTIADRKAHEAIVAILNE- |
| PACAP | 2P1N | LVPAYLKAVKQRRA |
| | | AVSTYDTFIGDSH |
| PYY | 4YIG | YRQASSADLTNLKELLSL |
| | | YKSEPSADEGPAEPKIPY |
| FOX04 | 3K02 | LTLRKEPASRAERILQLI |
| | | EQYPQAGWANRRSGGKRP |
| | | PPRRRQRRKKRG |
| GRS | 3BZI | DVFYQKM |
| Glucagon | 2QJS | TNMLWQVFDQARRLTAYSR |
| | | RYDEILTGQSH |

### Figures Caption

FIG. 1 shows limitations of current (D)-protein engineering techniques and a new method. (a) Loss of specific peptide-target interactions as a consequence of direct conversion to (D)-amino acids in helical peptides. Charged groups are shown on the target (black) as a white ‘plus’ or ‘minus’ signs on dark grey or grey (respectively) spots. Peptide charges are shown as dark grey or grey plus and minus signs. Target hydrogen bond participating groups are shown as a white ‘H’ on a grey spot. Grey curly arrows highlight the change in helix handedness from right to left upon conversion to (D). (D)-helix left-handedness means that helical peptide-target interactions fail to be restored, even when subject to RI. (b) Histograms showing the distribution of protein target sizes (in residues) for FDA approved drugs (top) and drug candidates being investigated (bottom). Targets that meet current commercial size limits for (D)-target synthesis are dwarfed by those precluded from MIPD. (c) Schematic overview of the method presented herein. Hotspot residues constellations are used to search a (D) amino acid version of the PDB including ˜2.8 m helices. Matches bind to the (L)-target with comparable affinity.

FIG. 2 is a schematic illustrating (D)-PDB construction. Every PDB file is retrieved, some containing various non-peptide molecules such as nucleic acids (grey) and solvent (dark grey). These are removed before creating a mirror image of the remaining protein molecule Cartesian coordinates—resulting in helix handedness change from right to left (light grey arrows). More than 2.8 million (D)-helices are extracted into separate files. Example PDB file used is 1 nkp (Myc-DNA complex).

FIG. 3 shows the preparation of GLP-1 queries for scanning the (D)-PDB. (a) GLP-1 structures and sequence, including the free peptide in solution (left) and receptor ECD bound structure (right). Free GLP-1 was solved using NMR and reveals a central unstructured linker region in contrast to GLP-1R bound. Hotspot and junction residues are annotated in grey and black respectively. (b) Hotspot are extracted separately for helix one and two, together with junction residues that have their backbone atoms rotated 180°. Rotation ensures (D)-peptide matches have reversed sequence order—a requirement for RI linker and tail attachment. (c) Levels (1-3) are assigned to hotspot atom pairs or triplets according to estimated import to target binding. (d) Atom levels are combined with similar residues. A combination order of decreasing quality—to sequentially test the (D)-PDB until close matches are identified—is thereby established.

FIG. 4 shows GLP-1 Best (D)-match results and full (D)-peptide construction. (a) (L)-query sequences (top) showing hotspots (grey), junction residues (black), and remaining original sequence (grey). Closest matching (D) structures are shown with atom levels annotated with dots corresponding to colours from FIG. 3. Match sequences are significantly different to query sequences. Helix 1 is highlighted light grey and helix 2—dark grey. (b) Full (D)-analog construction from best (D) match helix sequences juxtaposed with retro-inverted (RI) linker and terminal tail sequences. (c) Construction of D-analog structure from match helices and modelled linker. Docking to GLP-1R ECD identifies potential steric clashes, circumvented by mutation to alanine. Re-introduction of native peptide side-chains at two junction positions is also judged prudent. (d) PSI-PRED predicts that correct secondary structure is maintained in the (D)-analog, with medium-to-high confidence (blue bars). (e) Solubility check results predict good solubility.

FIG. 5 shows activity and protease degradation of (L) and (D)-GLP1 peptides. (a) HEK293 cells stably expressing GLP1R and CRE-luciferase were stimulated with different concentrations of (L)-, (D)-GLP1 peptides and Forskolin. Luciferase activity was measured. The experiments were performed in triplicate. (b) HEK293 cells stably expressing GLP1R were stimulated with 10 μM of (L)- or (D)-GLP1 peptide at different time-points. Proteins were resolved by SDS-PAGE and Western blotted using anti-phospho-ERK1/2, anti-ERK1/2, anti-phospho-AKT or anti-AKT antibodies. The experiments were performed in triplicate. A representative blot is shown for each antibody. (c) Sample gel images of (L) and (D)-GLP-1 peptides treated with Proteinase K (ProtK) over 5 hours. Gels were stained with Coomassie Brilliant Blue dye and band densitometry calculated using ImageJ (40) with background subtraction. (d) Quantification of remaining peptide post ProtK treatment in 60 min intervals. Intensities of peptide bands were normalized to the intensity of the untreated peptide (T0) and converted to a percentage relative to T0. The (L)-enantiomeric form undergoes rapid degradation while the (D)-enantiomer persists after 5 hours of treatment with ProtK. Error bars are reporting standard error. Data represent the average of 3 independent experiments.

FIG. 6 shows construction, activity and protease degradation of (L)- and (D)-PTH peptides. (a) (L)-PTH structure and sequence with hotspots highlighted in grey and junctions in black. (b) (D)-PDB match structures and sequences. (c) Final (D)-PTH construction from match sequences and RI (d) HEK293 cells stably expressing PTH1R and CRE-luciferase were stimulated with different concentrations of (L)-, (D)-PTH peptides and Forskolin. Luciferase activity was measured. The experiments were performed in triplicate. (e) Sample gel images of (L)- and (D)-PTH peptides treated with Proteinase K (ProtK) over 5 hours. Gels were stained with Coomassie Brilliant Blue dye and band densitometry calculated using ImageJ (40) with background subtraction. (f) Quantification of remaining peptide post ProtK treatment in 50 min intervals. Intensity of peptide bands were normalized to the intensity of the untreated peptide (T0) and converted to a percentage relative to T0. The (L)-enantiomeric form undergoes rapid degradation while the (D)-enantiomer persists after 5 hours of treatment with ProtK. Error bars are reporting standard error. Data represent the average of 3 independent experiments.

FIG. 12 shows the following: (A) GLP2 structure and sequence. Free GLP-2 was solved using NMR and has two helices connected by an unstructured linker. PDB ID: 2L63. Hotspot and junction residues are annotated in grey and black, respectively. (B) each helix is cut out from the full peptide as a separate query. (C) (L)-query hotspot structures (grey) aligned with closest matching (D) structures from the D-PDB. Match sequences are significantly different to query sequences. Helix 1 match sequence is highlighted light grey and helix 2 is dark grey. (D) Full (D)-analog construction from best (D) match helix sequences juxtaposed with RI linker and terminal tail sequences. Val to Leu mutation restores the D-match hotspot residues to original query identities (D-GLP2_2).

FIG. 13 shows the following: (A) Relaxin chain B structure and sequence. Two-chain Relaxin was solved using X-ray crystallography at 1.5 Å. PDB ID: 6RLX. Chain B has one helix flanked by unstructured regions. Hotspot and junction residues are annotated in grey and black, respectively. (B) the helix is cut out from the full peptide as the query structure. (C) (L)-query hotspot structures (grey) aligned with closest matching (D) structures from the D-PDB. Match sequences are significantly different to query sequences. (D) Full (D)-analog construction from the best (D) match helix sequence flanked with RI terminal tail sequences. Three annotated mutations restore D-match hotspot residues to original query identities (D-RLN_2).

FIG. 14 is a series of charts and experiments showing the activity and protease degradation of (L)- and (D)-GLP2 polypeptides. (A) HEK293 cells stably expressing GLP2R and CRE-luciferase were stimulated with different concentrations of (L)- and (D)-GLP2 peptides. Luciferase activity was measured. The experiments were performed in triplicate. (B) Sample gel images of (L) and (D)-GLP-2 peptides treated with Proteinase K (ProtK) over 5 hours. Gels were stained with Coomassie Brilliant Blue dye and band densitometry calculated using ImageJ (40) with background subtraction. (C) Quantification of remaining peptide post ProtK treatment in 60 min intervals. Intensities of peptide bands were normalized to the intensity of the untreated peptide (T0) and converted to a percentage relative to T0. The (L)-enantiomeric form undergoes rapid degradation while the (D)-enantiomer persists after 5 hours of treatment with ProtK. Error bars are reporting standard error. Data represent the average of 3 independent experiments.

FIG. 15 is a series of charts and experiments showing the activity and protease degradation of (D)-RLN_1 (non-mutated) and (D)-RLN_2 (mutated) polypeptides. (A) HEK293 cells stably expressing RXFP1 and CRE-luciferase were stimulated with different concentrations of (D)-RLN_1 and (D)-RLN_2 peptides. Luciferase activity was measured. The experiments were performed in triplicate. (B) Sample gel images of (L) and (D)-RLN peptides treated with Proteinase K (ProtK) over 5 hours. Gels were stained with Coomassie Brilliant Blue dye and band densitometry calculated using ImageJ (40) with background subtraction. (C) Quantification of remaining peptide post ProtK treatment in 60 min intervals. Intensities of peptide bands were normalized to the intensity of the untreated peptide (T0) and converted to a percentage relative to T0. The (L)-enantiomeric form undergoes rapid degradation while the (D)-enantiomer persists after 5 hours of treatment with ProtK. Error bars are reporting standard error. Data represent the average of 3 independent experiments.

### REFERENCES

1. Bruno B J, Miller G D & Lim C S (2013) Basics and recent advances in peptide and protein drug delivery. Ther Deliv 4(11): 1443-1467.
2. Corbi-Verge C, Garton M, Nim S & Kim P M (2017) Strategies to develop inhibitors of motif-mediated protein-protein interactions as drug leads. Annu Rev Pharmacol Toxicol 57: 39-60.
3. Fosgerau K & Hoffmann T (2015) Peptide therapeutics: Current status and future directions. Drug Discov Today 20(1): 122-128.
4. Welch B D, VanDemark. AP, Heroux A, Hill C P & Kay M S (2007) Potent D-peptide inhibitors of HIV-1 entry. Proc Natl Acad Sci USA 104(43): 16828-16833.
5. Liu M, et al (2010) D-peptide inhibitors of the p53-MDM2 interaction for targeted molecular therapy of malignant neoplasms. Proc Natl Acad Sci USA 107(32): 14321-14326.
6. Kreil G (1997) D-amino acids in animal peptides. Annu Rev Biochem 66: 337-345.
7. Uppalapati M, et al (2016) A potent D-protein antagonist of VEGF-A is nonimmunogenic, metabolically stable, and longer-circulating in vivo. ACS Chem Biol 11(4): 1058-1065.
8. Rabideau A E & Pentelute B L (2015) A d-amino acid at the N-terminus of a protein abrogates its degradation by the N-end rule pathway. ACS Cent Sci 1(8): 423-430.
9. Nickl C K, et al (2010) (D)-amino acid analogues of DT-2 as highly selective and superior inhibitors of cGMP-dependent protein kinase ialpha. Biochim Biophys Acta 1804(3): 524-532.
10. Brugidou J, Legrand C, Mery J & Rabie A (1995) The retro-inverso form of a homeobox-derived short peptide is rapidly internalised by cultured neurones: A new basis for an efficient intracellular delivery system. Biochem Biophys Res Commun 214(2): 685-693.
11. Veine D M, Yao H, Stafford D R, Fay K S & Livant D L (2014) A D-amino acid containing peptide as a potent, noncovalent inhibitor of alpha5beta1 integrin in human prostate cancer invasion and lung colonization. Clin Exp Metastasis
12. Li C, et al (2010) Limitations of peptide retro-inverso isomerization in molecular mimicry. J Biol Chem 285(25): 19572-19581.
13. Raibaut L, Ollivier N & Melnyk 0 (2012) Sequential native peptide ligation strategies for total chemical protein synthesis. Chem Soc Rev 41(21): 7001-7015.
14. Law V, et al (2014) DrugBank 4.0: Shedding new light on drug metabolism. Nucleic Acids Res 42(D1): D1091-D1097.
15. Li C, et al (2013) Functional consequences of retro-inverso isomerization of a miniature protein inhibitor of the p53-MDM2 interaction. Bioorg Med Chem 21(14): 4045-4050.
16. Li H, et al (2015) Novel retro-inverso peptide inhibitor reverses angiotensin receptor autoantibody-induced hypertension in the rabbit. Hypertension 65(4): 793-799.
17. Ben-Yedidia T, Beignon A S, Partidos C D, Muller S & Arnon R (2002) A retro-inverso peptide analogue of influenza virus hemagglutinin B-cell epitope 91-108 induces a strong mucosal and systemic immune response and confers protection in mice after intranasal immunization. Mol Immunol 39(5-6): 323-331.
18. Hung L W, Kohmura M, Ariyoshi Y & Kim S H (1998) Structure of an enantiomeric protein, D-monellin at 1.8 A resolution. Acta Crystallogr D Biol Crystallogr 54(Pt 4): 494-500.
19. Novotny M & Kleywegt G J (2005) A survey of left-handed helices in protein structures. J Mol Biol 347(2): 231-241.
20. Jochim A L & Arora P S (2010) Systematic analysis of helical protein interfaces reveals targets for synthetic inhibitors. ACS Chem Biol 5(10): 919-923.
21. Schumacher T N, et al (1996) Identification of D-peptide ligands through mirror-image phage display. Science 271(5257): 1854-1857.
22. Weinstock M T, Jacobsen M T & Kay M S (2014) Synthesis and folding of a mirror-image enzyme reveals ambidextrous chaperone activity. Proc Natl Acad Sci USA 111(32): 11679-11684.
23. Overington J P, Al-Lazikani B & Hopkins A L (2006) How many drug targets are there?. Nat Rev Drug Discov 5(12): 993-996.
24. Rudiger S, Schneider-Mergener J & Bukau B (2001) Its substrate specificity characterizes the DnaJ co-chaperone as a scanning factor for the DnaK chaperone. EMBO J 20(5): 1042-1050.
25. Manandhar B & Ahn J M (2015) Glucagon-like peptide-1 (GLP-1) analogs: Recent advances, new possibilities, and therapeutic implications. J Med Chem 58(3): 1020-1037.
26. Underwood C R, et al (2010) Crystal structure of glucagon-like peptide-1 in complex with the extracellular domain of the glucagon-like peptide-1 receptor. J Biol Chem 285(1): 723-730.
27. Buchan D W, Minneci F, Nugent T C, Bryson K & Jones D T (2013) Scalable web services for the PSIPRED protein analysis workbench. Nucleic Acids Res 41(Web Server issue): W349-57.
28. Lear S & Cobb S L (2016) Pep-calc.com: A set of web utilities for the calculation of peptide and peptoid properties and automatic mass spectral peak assignment. J Comput Aided Mol Des 30(3): 271-277.
29. Dunbar R L, et al (2017) Oral apolipoprotein A-I mimetic D-4F lowers HDL-inflammatory index in high-risk patients: A first-in-human multiple-dose, randomized controlled trial. Clin Transl Sci
30. Bloedon L T, et al (2008) Safety, pharmacokinetics, and pharmacodynamics of oral apoA-I mimetic peptide D-4F in high-risk cardiovascular patients. J Lipid Res 49(6): 1344-1352.
31. Rubin M R, et al (2016) Therapy of hypoparathyroidism with PTH(1-84): A prospective six year investigation of efficacy and safety. J Clin Endocrinol Metab 101(7): 2742-2750.
32. Della Rocca G J, Crist B D & Murtha Y M (2010) Parathyroid hormone: Is there a role in fracture healing?. J Orthop Trauma 24 Suppl 1: S31-5.
33. Dean T, Khatri A, Potetinova Z, Willick G E & Gardella T J (2006) Role of amino acid side chains in region 17-31 of parathyroid hormone (PTH) in binding to the PTH receptor. J Biol Chem 281(43): 32485-32495.
34. Pioszak A A & Xu H E (2008) Molecular recognition of parathyroid hormone by its G protein-coupled receptor. Proc Natl Acad Sci USA 105(13): 5034-5039.
35. Widmann C, Dolci W & Thorens B (1995) Agonist-induced internalization and recycling of the glucagon-like peptide-1 receptor in transfected fibroblasts and in insulinomas. Biochem J 310 (Pt 1)(Pt 1): 203-214.
36. Roed S N, et al (2015) Functional consequences of glucagon-like peptide-1 receptor cross-talk and trafficking. J Biol Chem 290(2): 1233-1243.
37. Qvit N, Rubin S J, Urban T J, Mochly-Rosen D & Gross E R (2017) Peptidomimetic therapeutics: Scientific approaches and opportunities. Drug Discov Today 22(2): 454-462.
38. Nguyen M N, Tan K P & Madhusudhan M S (2011) CLICK-topology-independent comparison of biomolecular 3D structures. Nucleic Acids Res 39(Web Server issue): W24-8.
39. Pettersen E F, et al (2004) UCSF chimera—a visualization system for exploratory research and analysis. J Comput Chem 25(13): 1605-1612.
40. Schneider C A, Rasband W S & Eliceiri K W (2012) NIH image to ImageJ: 25 years of image analysis. Nat Methods 9(7): 671-675.

1. A method for designing in-silico a (D)-polypeptide ligand that binds with a target, the method comprising:
providing a (L)-polypeptide ligand that binds with the target, the (L)-polypeptide ligand comprising one or more (L)-helical region;
for each of the one or more (L)-helical region:
identifying hotspot residues of the (L)-helical region, that interact with residues of the target; and
scanning a (D)-polypeptide database comprising single helix (D)-polypeptide candidates, to determine a single helix (D)-polypeptide match having a residue configuration that matches the hotspot residues of the (L)-helical region; and
generating the (D)-polypeptide ligand by combining the single helix (D)-polypeptide match of each of the one or more (L)-helical region.

2. The method of claim 1, wherein the (D)-polypeptide database is obtained by:
generating a mirror image of a (L)-polypeptide database to obtain a parallel polypeptide database; and
extracting the single helix (D)-polypeptide candidates from the parallel polypeptide database by trimming helical regions and removing non-helical parts from the parallel polypeptide database.

3. The method of claim 1 or 2, wherein the (D)-polypeptide match is determined by structural alignment of the residue configuration with the hotspot residues of the one or more (L)-helical region.

4. The method of any one of claims 1 to 3, wherein the (L)-polypeptide ligand further comprises one or more (L)-unstructured region, and wherein generating the (D)-polypeptide ligand is performed by combining the single helix (D)-polypeptide match of each of the one or more (L)-helical region and a (D)-retro-inverted version of each of the one or more (L)-unstructured region.

5. The method of claim 4, further comprising:
for each of the one or more (L)-helical region:
identifying junction residues located at a junction of a (L)-unstructured region and the (L)-helical region;
positioning the backbone of the junction residues, comprising for each junction residue:
performing a first rotation between 170° and 190° about the Cα-Cβ bond axis of the junction residue; and
performing a second rotation between 98.5° and 118.5° about the Cα of the junction residue, such that Cα-R and Cα-H exchange positions.

6. The method of claim 5, wherein junction residues are immediately adjacent to the one ore more (L)-unstructured region.

7. The method of claim 5 or 6, wherein the first rotation is a 180° rotation.

8. The method of any one of claims 5 to 7, wherein the second rotation is a 108.5° rotation.

9. The method of any one of claims 1 to 8, wherein the hotspot residues are identified on a (L)-polypeptide ligand conformation corresponding to the (L)-polypeptide ligand bound to the target.

10. The method of any one of claims 1 to 9, further comprising:
for each of the one or more (L)-helical region: generating a query library of (L)-query helices by mutating one or more hotspot residues,
wherein the single helix (D)-polypeptide match is determined by comparing the residue configuration with the hotspot residues of the one or more (L)-query helices.

11. The method of any one of claims 1 to 10, further comprising mutating the (D)-polypeptide ligand to increase binding affinity with the target.

12. A method for designing in silico a (D)-polypeptide ligand that binds with a target, the method comprising:
providing a (L)-polypeptide ligand that binds with the target, the (L)-polypeptide ligand comprising one or more (L)-helical region;
for each of the one or more (L)-helical region:
identifying hotspot residues of the (L)-helical region, that interact with residues of the target;
providing a (D)-mirror image of the one or more (L)-helical region;
scanning a (L)-polypeptide database comprising single helix (L)-polypeptide candidates, to determine a single helix (L)-polypeptide match having a residue configuration that matches the hotspot residues of the (D)-mirror image of the (L)-helical region; and
generating a (D)-mirror image of the single helix (L)-polypeptide match; and
generating the (D)-polypeptide ligand by combining the (D)-mirror image of the single helix (L)-polypeptide match of each of the one or more (L)-helical region.

13. The method of claim 12, wherein the (L)-polypeptide database is obtained by extracting single helix (L)-polypeptide candidates from a protein data bank.

14. The method of claim 12 or 13, wherein the (L)-polypeptide match is determined by structural alignment of the residue configuration with the hotspot residues of the (D)-mirror image of the one or more (L)-helical region.

15. The method of any one of claims 12 to 14, wherein the (L)-polypeptide ligand further comprises one or more (L)-unstructured region, and wherein generating the (D)-polypeptide ligand is performed by combining the (D)-mirror image of the single helix (L)-polypeptide match of each of the one or more (L)-helical region and a (D)-retro-inverted version of each of the one or more (L)-unstructured region.

16. The method of claim 15, further comprising:
for each of the one or more (L)-helical region:
identifying junction residues located at the junction of a (L)-unstructured region and the (L)-helical region;
positioning the backbone of the junction residues, comprising for each junction residue:
performing a first rotation between 170° and 190° about the Cα-Cβ bond axis of the junction residue; and
performing a second rotation between 98.5° and 118.5° about the Cα of the junction residue, such that Cα-R and Cα-H exchange positions.

17. The method of claim 16, wherein junction residues are immediately adjacent to the one ore more (L)-unstructured region.

18. The method of claim 16 or 17, wherein the first rotation is a 180° rotation.

19. The method of any one of claims 16 to 18, wherein the second rotation is a 108.5° rotation.

20. The method of any one of claims 12 to 19, wherein the hotspot residues are identified on a (L)-polypeptide ligand conformation corresponding to the (L)-polypeptide ligand bound to the target.

21. The method of any one of claims 12 to 20, further comprising:
for each of the one or more (L)-helical region: generating a query library of (L)-query helices by mutating one or more hotspot residues,
wherein providing a (D)-mirror image of the one or more (L)-helical region comprises providing a (D)-query helices that are (D)-mirror images of the (L)-query helices,
wherein the single helix (L)-polypeptide match is determined by comparing the residue configuration with the hotspot residues of the one or more (D)-query helices.

22. The method of any one of claims 12 to 21, further comprising mutating the (D)-polypeptide ligand to increase binding affinity with the target and/or improve receptor activation.

23. A method for designing in-silico a (D)-polypeptide ligand that binds with a target, the method comprising:
providing a (L)-polypeptide ligand that binds with the target, the (L)-polypeptide ligand comprising one or more (L)-helical region;
for each of the one or more (L)-helical region:
scanning a (D)-polypeptide database comprising single helix (D)-polypeptide candidates, to determine a single helix (D)-polypeptide match that matches the (L)-helical region; and
generating the (D)-polypeptide ligand by combining the single helix (D)-polypeptide match of each of the one or more (L)-helical region.

24. The method of claim 23, wherein the (D)-polypeptide database is obtained by:
generating a mirror image of a (L)-polypeptide database to obtain a parallel polypeptide database; and
extracting the single helix (D)-polypeptide candidates from the parallel polypeptide database by trimming helical regions and removing non-helical parts from the parallel polypeptide database.

25. The method of claim 23 or 24, further comprising: for each of the one or more (L)-helical region: identifying hotspot residues of the (L)-helical region, that interact with residues of the target, wherein scanning the (D)-polypeptide database allows to determine a single helix (D)-polypeptide match having a residue configuration that matches the hotspot residues of the (L)-helical region.

26. The method of claim 25, wherein the (D)-polypeptide match is determined by structural alignment of the residue configuration with the hotspot residues of the one or more (L)-helical region.

27. The method of claim 25 or 26, wherein the hotspot residues are identified on a (L)-polypeptide ligand conformation corresponding to the (L)-polypeptide ligand bound to the target.

28. The method of any one of claims 25 to 27, further comprising:
for each of the one or more (L)-helical region: generating a query library of (L)-query helices by mutating one or more hotspot residues,
wherein the single helix (D)-polypeptide match is determined by comparing the residue configuration with the hotspot residues of the one or more (L)-query helices.

29. The method of any one of claims 23 to 28, wherein the (L)-polypeptide ligand further comprises one or more (L)-unstructured region, and wherein generating the (D)-polypeptide ligand is performed by combining the single helix (D)-polypeptide match of each of the one or more (L)-helical region and a (D)-retro-inverted version of each of the one or more (L)-unstructured region.

30. The method of claim 29, further comprising:
for each of the one or more (L)-helical region:
identifying junction residues located at a junction of a (L)-unstructured region and the (L)-helical region;
positioning the backbone of the junction residues, comprising for each junction residue:
performing a first rotation between 170° and 190° about the Cα-Cβ bond axis of the junction residue; and
performing a second rotation between 98.5° and 118.5° about the Cα of the junction residue, such that Cα-R and Cα-H exchange positions.

31. The method of claim 30, wherein junction residues are immediately adjacent to the one ore more (L)-unstructured region.

32. The method of claim 30 or 31, wherein the first rotation is a 180° rotation.

33. The method of any one of claims 30 to 32, wherein the second rotation is a 108.5° rotation.

34. The method of any one of claims 23 to 33, further comprising mutating the (D)-polypeptide ligand to increase binding affinity with the target.

35. A method for designing in silico a (D)-polypeptide ligand that binds with a target, the method comprising:
providing a (L)-polypeptide ligand that binds with the target, the (L)-polypeptide ligand comprising one or more (L)-helical region;
for each of the one or more (L)-helical region:
providing a (D)-mirror image of the (L)-helical region;
scanning a (L)-polypeptide database comprising single helix (L)-polypeptide candidates, to determine a single helix (L)-polypeptide match that matches the (D)-mirror image of the (L)-helical region; and
generating a (D)-mirror image of the single helix (L)-polypeptide match; and
generating the (D)-polypeptide ligand by combining the (D)-mirror image of the single helix (L)-polypeptide match of each of the one or more (L)-helical region.

36. The method of claim 35, wherein the (L)-polypeptide database is obtained by extracting single helix (L)-polypeptide candidates from a protein data bank.

37. The method of claim 35 or 36, further comprising: for each of the one or more (L)-helical region: identifying hotspot residues of the (L)-helical region, that interact with residues of the target, wherein scanning the (L)-polypeptide database allows to determine a single helix (L)-polypeptide match having a residue configuration that matches the hotspot residues of the (D)-mirror image of the (L)-helical region.

38. The method of claim 37, wherein the hotspot residues are identified on a (L)-polypeptide ligand conformation corresponding to the (L)-polypeptide ligand bound to the target.

39. The method of claim 37 or 38, further comprising:
for each of the one or more (L)-helical region: generating a query library of (L)-query helices by mutating one or more hotspot residues,
wherein providing a (D)-mirror image of the one or more (L)-helical region comprises providing a (D)-query helices that are (D)-mirror images of the (L)-query helices,
wherein the single helix (L)-polypeptide match is determined by comparing the residue configuration with the hotspot residues of the one or more (D)-query helices.

40. The method of any one of claims 37 to 39, wherein the (L)-polypeptide match is determined by structural alignment of the residue configuration with the hotspot residues of the (D)-mirror image of the one or more (L)-helical region.

41. The method of any one of claims 35 to 40, wherein the (L)-polypeptide ligand further comprises one or more (L)-unstructured region, and wherein generating the (D)-polypeptide ligand is performed by combining the (D)-mirror image of the single helix (L)-polypeptide match of each of the one or more (L)-helical region and a (D)-retro-inverted version of each of the one or more (L)-unstructured region.

42. The method of claim 41, further comprising:
for each of the one or more (L)-helical region:
identifying junction residues located at the junction of a (L)-unstructured region and the (L)-helical region;
positioning the backbone of the junction residues, comprising for each junction residue:
performing a first rotation between 170° and 190° about the Cα-Cβ bond axis of the junction residue; and
performing a second rotation between 98.5° and 118.5° about the Cα of the junction residue, such that Cα-R and Cα-H exchange positions.

43. The method of claim 42, wherein junction residues are immediately adjacent to the one ore more (L)-unstructured region.

44. The method of claim 42 or 43, wherein the first rotation is a 180° rotation.

45. The method of any one of claims 42 to 44, wherein the second rotation is a 108.5° rotation.

46. The method of any one of claims 35 to 45, further comprising mutating the (D)-polypeptide ligand to increase binding affinity with the target and/or improve receptor activation.

47. The method of any one of claims 1 to 46, wherein the target is a (L)-polypeptide target.

48. The method of any one of claims 1 to 47, wherein the target is a GLP-1 receptor (GLP1R).

49. The method of any one of claims 1 to 47, wherein the target is a PTH receptor (PTH1R).

50. The method of any one of claims 1 to 47, wherein the target is a GLP-2 receptor (GLP2R).

51. The method of any one of claims 1 to 47, wherein the target is a Relaxin (RLN) receptor.

52. A (D)-analog of GLP-1, comprising a (D)-amino acid sequence having a sequence identity of 80% or greater to the sequence of SEQ ID NO:1.

53. The (D)-analog of claim 52, comprising a (D)-amino acid sequence having a sequence identity of 90% or greater to the sequence of SEQ ID NO:1.

54. The (D)-analog of claim 52, comprising a (D)-amino acid sequence having a sequence identity of 95% or greater to the sequence of SEQ ID NO:1.

55. The (D)-analog of claim 52, comprising a (D)-amino acid sequence as shown in the sequence of SEQ ID NO:1.

56. Use of the (D)-analog of any one of claims 52 to 55, for the treatment or prevention of diabetes.

57. Use of the (D)-analog of any one of claims 52 to 55, for the treatment of diabetes.

58. Use of the (D)-analog of any one of claims 52 to 55, for the treatment or prevention of obesity.

59. Use of the (D)-analog of any one of claims 52 to 55, for the treatment of obesity.

60. A (D)-analog of PTH, comprising a (D)-amino acid sequence having a sequence identity of 80% or greater to the sequence of SEQ ID NO:2.

61. The (D)-analog of claim 60, comprising a (D)-amino acid sequence having a sequence identity of 90% or greater to the sequence of SEQ ID NO:2.

62. The (D)-analog of claim 60, comprising a (D)-amino acid sequence having a sequence identity of 95% or greater to the sequence of SEQ ID NO:2.

63. The (D)-analog of claim 60, comprising a (D)-amino acid sequence as shown in the sequence of SEQ ID NO:2.

64. Use of the (D)-analog of any one of claims 60 to 63, for the treatment or prevention of osteoporosis.

65. Use of the (D)-analog of any one of claims 60 to 63, for the treatment of osteoporosis.

66. Use of the (D)-analog of any one of claims 60 to 63, for the treatment or prevention of hyperparathyroidism

67. Use of the (D)-analog of any one of claims 60 to 63, for the treatment of hyperparathyroidism.

68. Use of the (D)-analog of any one of claims 60 to 63, for promoting bone growth.

69. A (D)-analog of GLP-2, comprising a (D)-amino acid sequence having a sequence identity of 80% or greater to the sequence of SEQ ID NO:3.

70. The (D)-analog of claim 69, comprising a (D)-amino acid sequence having a sequence identity of 90% or greater to the sequence of SEQ ID NO:3.

71. The (D)-analog of claim 69, comprising a (D)-amino acid sequence having a sequence identity of 95% or greater to the sequence of SEQ ID NO:3.

72. The (D)-analog of claim 69, comprising a (D)-amino acid sequence as shown in the sequence of SEQ ID NO:3.

73. Use of the (D)-analog of any one of claims 69 to 72, for the treatment or prevention of a gastrointestinal disease.

74. Use of the (D)-analog of any one of claims 69 to 72, for the treatment of a gastrointestinal disease.

75. Use of the (D)-analog of any one of claims 69 to 72, for the treatment or prevention of obesity.

76. Use of the (D)-analog of any one of claims 69 to 72, for the treatment of obesity.

77. Use of the (D)-analog of any one of claims 69 to 72, for the treatment or prevention of metabolic endotoxemia.

78. Use of the (D)-analog of any one of claims 69 to 72, for the treatment of metabolic endotoxemia.

79. Use of the (D)-analog of any one of claims 69 to 72, for the treatment or prevention of short bowel syndrome (SBS).

80. Use of the (D)-analog of any one of claims 69 to 72, for the treatment of short bowel syndrome (SBS).

81. Use of the (D)-analog of any one of claims 69 to 72, for the treatment or prevention of diabetes.

82. Use of the (D)-analog of any one of claims 69 to 72, for the treatment of diabetes.

83. A (D)-analog of Relaxin (RLN), comprising a (D)-amino acid sequence having a sequence identity of 80% or greater to the sequence of SEQ ID NO:4.

84. The (D)-analog of claim 83, comprising a (D)-amino acid sequence having a sequence identity of 90% or greater to the sequence of SEQ ID NO:4.

85. The (D)-analog of claim 83, comprising a (D)-amino acid sequence having a sequence identity of 95% or greater to the sequence of SEQ ID NO:4.

86. The (D)-analog of claim 83, comprising a (D)-amino acid sequence as shown in the sequence of SEQ ID NO:4.

87. Use of the (D)-analog of any one of claims 83 to 86, for the treatment or prevention of fibrosis.

88. Use of the (D)-analog of any one of claims 83 to 86, for the treatment of fibrosis.

89. Use of the (D)-analog of any one of claims 83 to 86, for the treatment or prevention of inflammation.

90. Use of the (D)-analog of any one of claims 83 to 86, for the treatment of inflammation.

91. Use of the (D)-analog of any one of claims 83 to 86, for cardioprotection.

92. Use of the (D)-analog of any one of claims 83 to 86, for the treatment or prevention of vasodilatation.

93. Use of the (D)-analog of any one of claims 83 to 86, for the treatment of vasodilatation.

94. Use of the (D)-analog of any one of claims 83 to 86, for enhancing angiogenesis.

95. A (D)-polypeptide, comprising a (D)-amino acid sequence having a sequence identity of 80% or greater to the sequence of SEQ ID NO:5.

96. The (D)-polypeptide of claim 95, comprising a (D)-amino acid sequence having a sequence identity of 90% or greater to the sequence of SEQ ID NO:5.

97. The (D)-polypeptide of claim 95, comprising a (D)-amino acid sequence having a sequence identity of 95% or greater to the sequence of SEQ ID NO:5.

98. The (D)-polypeptide of claim 95, comprising a (D)-amino acid sequence as shown in the sequence of SEQ ID NO:5.

99. A (D)-polypeptide, comprising a (D)-amino acid sequence having a sequence identity of 80% or greater to the sequence of SEQ ID NO:6.

100. The (D)-polypeptide of claim 99, comprising a (D)-amino acid sequence having a sequence identity of 90% or greater to the sequence of SEQ ID NO:6.

101. The (D)-polypeptide of claim 99, comprising a (D)-amino acid sequence having a sequence identity of 95% or greater to the sequence of SEQ ID NO:6.

102. The (D)-polypeptide of claim 99, comprising a (D)-amino acid sequence as shown in the sequence of SEQ ID NO:6.

103. A (D)-polypeptide, comprising a (D)-amino acid sequence having a sequence identity of 80% or greater to the sequence of SEQ ID NO:7.

104. The (D)-polypeptide of claim 103, comprising a (D)-amino acid sequence having a sequence identity of 90% or greater to the sequence of SEQ ID NO:7.

105. The (D)-polypeptide of claim 103, comprising a (D)-amino acid sequence having a sequence identity of 95% or greater to the sequence of SEQ ID NO:7.

106. The (D)-polypeptide of claim 103, comprising a (D)-amino acid sequence as shown in the sequence of SEQ ID NO:7.

107. A (D)-polypeptide, comprising a (D)-amino acid sequence having a sequence identity of 80% or greater to the sequence of SEQ ID NO:8.

108. The (D)-polypeptide of claim 107, comprising a (D)-amino acid sequence having a sequence identity of 90% or greater to the sequence of SEQ ID N0:8.

109. The (D)-polypeptide of claim 107, comprising a (D)-amino acid sequence having a sequence identity of 95% or greater to the sequence of SEQ ID N0:8.

110. The (D)-polypeptide of claim 107, comprising a (D)-amino acid sequence as shown in the sequence of SEQ ID NO:8.

111. A (D)-polypeptide, comprising a (D)-amino acid sequence having a sequence identity of 80% or greater to the sequence of SEQ ID NO:9.

112. The (D)-polypeptide of claim 111, comprising a (D)-amino acid sequence having a sequence identity of 90% or greater to the sequence of SEQ ID N0:9.

113. The (D)-polypeptide of claim 111, comprising a (D)-amino acid sequence having a sequence identity of 95% or greater to the sequence of SEQ ID N0:9.

114. The (D)-polypeptide of claim 111, comprising a (D)-amino acid sequence as shown in the sequence of SEQ ID NO:9.

115. A (D)-polypeptide, comprising a (D)-amino acid sequence having a sequence identity of 80% or greater to the sequence of SEQ ID NO:10.

116. The (D)-polypeptide of claim 115, comprising a (D)-amino acid sequence having a sequence identity of 90% or greater to the sequence of SEQ ID NO:10.

117. The (D)-polypeptide of claim 115, comprising a (D)-amino acid sequence having a sequence identity of 95% or greater to the sequence of SEQ ID NO:10.

118. The (D)-polypeptide of claim 115, comprising a (D)-amino acid sequence as shown in the sequence of SEQ ID NO:10.

119. A (D)-polypeptide, comprising a (D)-amino acid sequence having a sequence identity of 80% or greater to the sequence of SEQ ID NO:11.

120. The (D)-polypeptide of claim 119, comprising a (D)-amino acid sequence having a sequence identity of 90% or greater to the sequence of SEQ ID NO:11.

121. The (D)-polypeptide of claim 119, comprising a (D)-amino acid sequence having a sequence identity of 95% or greater to the sequence of SEQ ID NO:11.

122. The (D)-polypeptide of claim 119, comprising a (D)-amino acid sequence as shown in the sequence of SEQ ID NO:11.

123. A (D)-polypeptide, comprising a (D)-amino acid sequence having a sequence identity of 80% or greater to the sequence of SEQ ID NO:12.

124. The (D)-polypeptide of claim 123, comprising a (D)-amino acid sequence having a sequence identity of 90% or greater to the sequence of SEQ ID NO:12.

125. The (D)-polypeptide of claim 123, comprising a (D)-amino acid sequence having a sequence identity of 95% or greater to the sequence of SEQ ID NO:12.

126. The (D)-polypeptide of claim 123, comprising a (D)-amino acid sequence as shown in the sequence of SEQ ID NO:12.

127. A compound, that is obtained by the method of any one of claims 1 to 51.

128. A method for generating in-silico a (D)-polypeptide database, the method comprising:
generating a mirror image of a (L)-polypeptide database comprising (L)-polypeptides, to obtain a parallel polypeptide database comprising (D)-polypeptides mirror images of the (L)-polypeptides; and
extracting single helix (D)-polypeptides from the parallel polypeptide database, comprising trimming helical regions of the (D)-polypeptides and removing non-helical regions from the parallel polypeptide database, to obtain the (D)-polypeptide database.

129. The method of claim 128, wherein the (D)-polypeptide database consists of the single helix (D)-polypeptides.

130. The method of claim 128 or 129, wherein the (L)-polypeptide database comprises the Protein Data Bank (PDB).

131. A (D)-polypeptide database, obtained by the method of any one of claims 128 to 130.

132. A (D)-polypeptide database, consisting of single helix (D)-polypeptides obtained by generating a mirror image of a (L)-polypeptide database comprising (L)-polypeptides to obtain (D)-polypeptides; and extracting the single helix (D)-polypeptides from the parallel (D)-polypeptides by trimming helical regions and non-helical regions from the (D)-polypeptides, discarding the non-helical regions and storing the helical regions as the single helix (D)-polypeptides of the (D)-polypeptide database.


SEQUENCE LISTING (12 sequences)

SEQ ID NO: 1 - protein, 29 aa, Artificial Sequence
  (D)-analog of GLP-1 - Sequence consisting of (D)-amino acids
RGKAFLELFILAAGELIEAIDRTFTGEAH

SEQ ID NO: 2 - protein, 36 aa, Artificial Sequence
  (D)-analog of PTH - Sequence consisting of (D)-amino acids
AGLAVADSVGMLLMYLPEWRNHKGDLMREIRSRKVS

SEQ ID NO: 3 - protein, 32 aa, Artificial Sequence
  (D)-analog of GLP-2 - Sequence consisting of (D)-amino acids
DTIKTDRIGFWFAVAAILVLLKMMEEFVGDAH

SEQ ID NO: 4 - protein, 29 aa, Artificial Sequence
  (D)-analog of Relaxin - Sequence consisting of (D)-amino acids
SWTSKELLIEIINDLQKRGCLKIVEEMWS

SEQ ID NO: 5 - protein, 42 aa, Artificial Sequence
  (D)-ligand for Zika Virus - Sequence consisting of (D)-amino acids
REEVYEIFHAQHGTVRSLAFLATVSGFFERVWTGEMVVVAVC

SEQ ID NO: 6 - protein, 10 aa, Artificial Sequence
  (D)-ligand for Dengue Virus 1 of 2 - Sequence consisting of (D)-amino acids
DVDKTWDEYQ

SEQ ID NO: 7 - protein, 20 aa, Artificial Sequence
  (D)-ligand for Dengue Virus 2 of 2 - Sequence consisting of (D)-amino acids
SPLTIADRKAHEAIVAILNE

SEQ ID NO: 8 - protein, 27 aa, Artificial Sequence
  (D)-ligand for PACAP - Sequence consisting of (D)-amino acids
LVPAYLKAVKQRRAAVSTYDTFIGDSH

SEQ ID NO: 9 - protein, 36 aa, Artificial Sequence
  (D)-ligand for PYY - Sequence consisting of (D)-amino acids
YRQASSADLTNLKELLSLYKSEPSADEGPAEPKIPY

SEQ ID NO: 10 - protein, 48 aa, Artificial Sequence
  (D)-ligand for FOXO4 - Sequence consisting of (D)-amino acids
LTLRKEPASRAERILQLIEQYPQAGWANRRSGGKRPPPRRRQRRKKRG

SEQ ID NO: 11 - protein, 7 aa, Artificial Sequence
  (D)-ligand for GRS - Sequence consisting of (D)-amino acids
DVFYQKM

SEQ ID NO: 12 - protein, 30 aa, Artificial Sequence
  (D)-ligand for Glucagon - Sequence consisting of (D)-amino acids
TNMLWQVFDQARRLTAYSRRYDEILTGQSH